(19) United States
(12) Patent Application Publication
(10) Pub. No.: US 2010/0159507 A1
(43) Pub. Date: Jun. 24, 2010

(54) SHREDDER FOR MECHANICAL DISRUPTION BY GENTLE CONTROLLED COMPRESSIVE ROTATION

(76) Inventors: Edmund Y. Ting, Kent, WA (US); Alexander Lazarev, Lexington, MA (US); Vera Gross, Newtonville, MA (US); Charles Dussault, Taunton, MA (US); Chunqin Li, Rockville, MD (US); Nathan Lawrence, South Easton, MA (US); Richard Schumacher, Taunton, MA (US)

Correspondence Address: LANDO & ANASTASI, LLP, ONE MAIN STREET, SUITE 1100, CAMBRIDGE, MA, 02142, US

(21) Appl. No.: 12/562,098

(22) Filed: Sep. 17, 2009

Related U.S. Application Data
(60) Provisional application No. 61/097,830, filed on Sep. 17, 2008.

(51) Int. Cl. G01N 1/28; C12M 1/00; C12M 1/33

(52) U.S. Cl. 435/40.5; 435/283.1; 435/306.1

(57) ABSTRACT

The systems and techniques of the present invention can also synergistically utilize mechanical disruption processes with the use of high hydrostatic pressure extraction, such as pressure cycling extraction techniques to achieve high yield of difficult to extract sample constituents without generating high shear stress or high temperatures.

### CROSS-REFERENCE TO RELATED APPLICATION

This application claims the benefit of priority to U.S. Patent Application No. 61/097,830, titled SHREDDER FOR MECHANICAL DISRUPTION BY GENTLE CONTROLLED COMPRESSIVE ROTATION, filed on Sep. 17, 2009, the entire content of which is incorporated herein by reference for all purposes.

### BACKGROUND

1. Field

The present disclosure is directed to providing and preparing samples for analysis thereof and in particular to preparing biological samples to facilitate extraction and analysis of small molecules such as deoxyribonucleic acid, ribonucleic acid, lipid, protein by shredding the biological samples under disruptive forces created by rotationally directed forces.

2. Related Art

The extraction of DNA, RNA, protein, lipid, and small molecule from biological samples is made difficult when these samples have a tough external structure. For many sample types, if their tough external structures can be opened, pressure cycling technology (PCT) can be effectively used for sample extraction. PCT alone is a highly effective extraction method for cells with low strength membranes but in some instances may not be sufficient to break a tough external structure quickly. Examples of samples that are difficult to extract are plants seeds, whole insects, and fibrous tissues.

Schumacher in U.S. Patent Application Publication No. 2002/0197631 discloses a barrier in chamber and forces a sample through barrier.

Saghbini in U.S. Patent Application Publication No. 2004/0005608 discloses rotatively mounted crusher rods, sample well support means, and a filter. Unlike some embodiments of the present invention, Saghbini disclosure does not provide control of force applied during rotation may not be compatible with pressure cycling techniques. Also unlike some embodiments of the present invention, no controlled processing is provided, which can generate undesirably excessive shearing condition that can damage some molecules or compounds of interest, such DNA and/or RNA or proteins.

Yamamoto in U.S. Patent Application Publication No. 2006/0078474 discloses centrifugally loaded member against supported filter. Unlike some embodiments of the present invention, Yamamoto's approach does not provide control of force applied during rotation and is not compatible with pressure cycling techniques. Like Saghbini, Yamamoto disclosure does not reduce the likelihood of over-processing which can damage sample content.

The previous attempts at sample extraction have typically focused on the use of pressure cycling alone or the use of mechanical grinding alone. Compression action alone typically does not result in high extraction yields, as the sample may not be sufficiently disrupted, especially if the applied energy input is low.

Pressure cycling techniques can be more effective on highly compressible sample components, such as lipids and proteins, when the sample is comprised of biological material such as cells or tissues. In certain instances organisms or cells, however, are surrounded by poorly compressible materials, e.g., proteoglycans, mureins or chitins, exemplified by bacterial or fungal cell wall, exoskeletons of crustaceans and insects, spore coats, etc., for which pressure cycling techniques have little disruptive effect. Such materials may have to be disrupted or degraded by breaking covalent bonds that connect polymer strands together in a mesh-like structure. This can be accomplished by high-energy mechanical homogenization, ultrasonic cavitation, sonication, enzymatic digestion, vibrational bead beating, etc. These disruption techniques, however, may be unreliable or even unpredictable such as with respect to the level of disruption. For example, if low or insufficient force or energy is applied, disruption is typically incomplete and the effective yield of analyzable molecules is low; and if high or excessive force or energy is applied, high shear stress and heat generated can mechanically and thermally, or both, alter the target extraction product, and undesirably change the characteristics thereof such that the produced sample is converted to be of a composition that is no longer of interest because the product is not representative of the target material.

Further, pressure cycling processing of strongly enveloped samples, such as plants seeds, whole insects or certain organ tissues, typically requires numerous pressure cycles to extract target compounds such as protein and DNA.

The devices of the present invention, or components thereof, can be comprised of a polymeric material, a mixture or blend of polymers, a metal, or a metal alloy.

### SUMMARY

One or more aspects of the invention can be directed to a device for sample processing, the device can comprise a container, a rotatable element at least partially disposed within the container and having a coupling end. The device can further have a smooth perforated divider disposed within the container. The perforated divider typically has a plurality of apertures therethrough. The perforated divider can have a smooth surface that is free of protrusions or depressions. The perforated divider can have surface features such as any one or more of serrations which can be uniformly sized or be of varying heights, perforations of various sizes, and teeth, which can be uniformly sized or be of varying heights and widths. The rotatable element can also have a surface that is typically exposed to a sample. The surface can be any of smooth, relatively free of surface asperities, teeth, which can be uniformly sized or be of varying heights and widths. The rotatable element can also have serrations protruding from the surface. The rotatable element can also be displaceable along a longitudinal axis of the container. The rotatable element can also serve as a ram by being displaceable along the longitudinal axis of the container. In some configurations, the rotatable element has a protrusions extending from a facial surface end, the protrusions sized to secure the sample against the element during rotational, axial, or rotational and axial translation of the rotatable element in the container. The device can also have a grinding surface disposed in the container, the grinding surface having asperities that serves as an abrasive surface against the sample during translation thereof resulting from the displacement of the rotatable element. The device can further comprise a seal disposed between a surface of an opening of the container and a surface of a shaft section of the rotatable element. The seal serving to fluidly isolate the internal volume within the container. The seal typically prevents fluidly from leaving the volume within the container. In some cases, the rotatable element can thus serve as a ramming component that, upon axial displacement thereof, pressurizes the internal volume of the container, preferably to a predetermined hydrostatic pressure. Axial displacement of the rotatable element, as a ramming component can be effected through an externally applied hydraulic or pneumatic forces. The device can comprise a lysis disk, such as those disclosed in pulse tubes from Pressure Biosciences, Inc., South Easton, Mass. The device can further comprise a spring-loaded surface. The spring-loaded surface is typically coupled at a spring-facing face thereof to a spring. In use, hydrostatic pressure applied to the contents of the container can compressively displace the spring. Linear displacement of the spring in response to the applied pressure is typically dependent on the spring constant, the magnitude of the pressure, and in some cases, the compressibility of the sample and other fluids in the container. The container is preferably a single use container which is disposed or destroyed after being charged with a first sample.

One or more aspects of the invention can be directed to a method of preparing a sample, the method comprising charging a sample into a sample container, and rotating a rotatable element having a surface thereof disposed against the sample. The method can further comprise applying a hydrostatic pressure on the sample within the container. In some cases, the applied hydrostatic pressure is generated by reducing the volume contained within the container, which in particular embodiments of the invention can be effected by axially displacing the rotatable element thereby compressing the container internal volume. The method can further comprise cooling the sample, preferably while contained in the sample container. In some further cases, the method can further comprise heating the sample, preferably while contained in the sample container. Heating the sample can be performed by exposing an external surface of the sample container to a heating environment. Cooling the sample can be performed by exposing the external surface of the sample container to a cooling environment. The method can also involve utilizing sample containers having a lysis disk disposed therein. Alternatively or in conjunction with the lysis disk, the method can involve charging abrasive media or grinding aids, such as balls into the sample container. In some of such cases, the method can further comprise agitating the sample within the sample container, for example, by utilizing a shaker device. Particular instances of the method can involve rotating the rotatable element. Further particular instances of the method can involve rotating the rotatable element at a predetermined rate of revolutions. For example, rotating can be performed at least one 1 revolution per minute (rpm), at least 10 rpm, at least 50 rpm, and even at least 100 rpm. In other exemplary instances, rotating can be performed at less than 500 rpm, but at least 200 rpm. The method can also involve cyclically rotating the rotatable element. Thus, in some cases, the method can involve rotating the rotatable element, such as at a first rotating rate and rotating the rotatable element at a second rotating rate. The elapsed period of the first rotating rate can be a first rotating period, and the period of the second rotating rate can be a second rotating period. The magnitude of the second rotating rate can be greater than the magnitude of the first rotating rate. The magnitude of the second rotating rate can be less than the magnitude of the first rotating rate. Rotating at the first rotating rate can be performed at a first rotational direction, and rotating at the second rotating rate can be performed at a second rotational direction that is opposite the first rotational direction. Rotating at the first rotating rate can be performed at a first rotational direction, and rotating at the second rotating rate can be performed at a second rotational direction that is the same as the first rotational direction. The second rotating period can be greater than the first rotating period. The second rotating period can be less than the first rotating period. Rotating to the first rotating rate can be effected at a first pace. For example, rotating to the first rotating rate can be performed within one second, within five seconds, or even within ten seconds. Rotating to the second rate can be effected at a second pace. The magnitude of the second pace or the elapsed time to achieve the second rotating rate can be the same as the magnitude of the first pace. The magnitude of the second pace or the elapsed time to achieve the second rotating rate can be greater than the magnitude of the first pace. The magnitude of the second pace or the elapsed time to achieve the second rotating rate can be less than the magnitude of the first pace.

The method can also involve rotating the rotatable element at a third rotating rate for a third rotating period. The first rotating rate and the third rotating rate can each have the same magnitude, and in some cases, the same period. In other cases, any of the first rotating rate, the second rotating rate, and the third rotating rate can be performed in directions that are relative opposite directions. The magnitude of the second rotating rate can be greater than the magnitude of the first rotating rate, the third rotating rate, or both. The magnitude of the third rotating rate can be greater than the magnitude of the first rotating rate, the second rotating rate, or both. The magnitude of the second rotating rate can be less than the magnitude of the first rotating rate, the third rotating rate, or both. The magnitude of the third rotating rate can be less than the magnitude of the first rotating rate, the second rotating rate, or both. The second rotating period can be greater than the first rotating period, the third rotating period, or both. The third rotating period can be greater than the first rotating period, the second rotating period, or both. The second rotating period can be less than the first rotating period, the third rotating period, or both. The third rotating period can be less than the first rotating period, the second rotating period, or both. Rotating to the first rotating rate can be effected at a first pace. For example, rotating to the first rotating rate can be performed within one second, within five seconds, or even within ten seconds. Rotating to the second rotating rate can be effected at a second pace. Rotating to the third rotating rate can be effected at a third pace. The magnitude of the second pace or the elapsed time to achieve the second rotating rate can be the same as the magnitude of the first pace, the third pace, or both. The magnitude of the second pace or the elapsed time to achieve the second rotating rate can be greater than the magnitude of the first pace, the third pace, or both. The magnitude of the second pace or the elapsed time to achieve the second rotating rate can be less than the magnitude of the first pace, the third pace, or both.

In accordance with some aspects, the present invention provides alternatives to or be utilized with high energy mechanical disruptive processes such as homogenization, ultrasonic cavitation, sonication, enzymatic digestion, and vibrational bead beating.

The systems and techniques of the present invention can also synergistically utilize mechanical disruption processes with the use of high hydrostatic pressure extraction, such as pressure cycling extraction techniques to achieve high yield of difficult to extract sample constituents without generating high shear stress or high temperatures. By using the mechanical shredding apparatus and processes of the invention to break the tough external structure of a sample, higher effective yields can be achieved in shorter periods, relative to conventional pressure cycling techniques because it is believed that by opening the external structure with mechanical pre-processing procedures, high-pressure fluid, such as those associated with pressure cycling techniques, can effectively permeate the external structures of a sample and thermodynamically loosen or release proteins and DNA for extraction. For example, conventional grinding can destroy or alter the natural characteristics of certain protein or DNA components, such as by denaturing. Further, grinding, such as by ultrasonic or bead milling, under high energy density conditions will create heat or high shear stresses, or both in some cases, resulting in damage or alteration of the proteins or DNA specimens.

### DESCRIPTION OF THE DRAWINGS

The accompanying drawings are not drawn to scale. In the drawings, each identical or nearly identical component or feature that is illustrated is represented by a like numeral. For purposes of clarity, not every component may be labeled. In the drawings:

FIG. 1 is a copy of a photograph of a shredder device that can be sample container in accordance with one or more aspects of the present invention;

FIG. 2 is a schematic illustration showing a cross-section of a shredder device that can be a sample container in accordance with one or more aspects of the present invention;

FIGS. 3A to 3D are schematic illustrations showing an assembly in which a shredder can be utilized in accordance with one or more aspects of the present invention, with FIG. 3A showing the shredder in the assembly, FIG. 3B showing a cross-sectional of the shredder in the assembly as well as copies of photographs of the assembly (noting a pressure settings and plant specimens disposed into the sample container, FIG. 3C schematically illustrates the rotational and axial translation of the rotatable element in the shredder device, and FIG. 3D schematically illustrates that shredder base assembly having the shredder device disposed therein;

FIGS. 4A to 4C are schematic illustrations of the shredder device with the base assembly, with FIG. 4A showing a perspective schematic view, FIG. 4B showing an elevational schematic view, and FIG. 4C showing a top plan view;

FIGS. 5A to 5C are further schematic illustrations of the shredder device and the shredder base assembly;

FIG. 6 is a copy of a photograph of a BIOMASHER™ device showing a homogenizer bar, an insert membrane, and a collection tube, in which the BIOMASHER device involves centrifugal homogenization of the sample as the sample material is driven by forces generated centrifugation through a porous membrane, or use with the homogenizer bar that is coupled to a rotating driver;

FIG. 7 is a graph showing the correlation between color of plant extracts and protein yields from pine needle samples extracted through pressure cycling techniques or use of BIOMASHER™ device, with ProteoSOLVE™ IEF reagent, the graph showing that tannins are reactive with Bradford reagent resulting in inflated protein values;

FIG. 8A shows graphs of a standard curve for relative estimation of total bacterial load (top two graphs and noted as picture 1) and of real time PCR on DNA preps from ticks isolated using the shredder techniques and devices of the present invention coupled with pressure cycling techniques (bottom two graphs and noted as picture 2);

FIG. 8B is a copy of a graph showing a real time PCR assay of Borrelia burgdorferi 23S rRNA gene which has been strongly amplified from tick DNA preps extracted by the shredder techniques and devices of the present invention coupled with lysing by pressure cycling techniques and devices, the graph showing identification in three of five as Ixodes scapilaris, and in two out of seven unidentified ticks having B. burgdorferi, the tick samples were effectively shredded in one minute (followed by pressure cycling processes) in the same tube or container;

FIG. 8C is a graph showing Ct values for E. coli (1×10⁷ cells) spiked tick samples (15.85 and 5.84) and Ct values for unspiked tick samples (18.96 and 18.09), in which significant Ct values for the unspiked tick samples indicating effective tick bacteria DNA isolations;

FIG. 8D are graphs showing (in the top two graphs and noted as picture 3) standard curves for relative quantification of Borrelia burgdorferi, the standard curve was set up using a set of primers and probe specific for amplification of B. burgdorferi 23S rRNA genes (Bb23Sr and Bb23Sf) for relative quantification of B. burgdorferi load (B. Burgdorferi DNA from ATCC was used as the standard for serial dilutions), and showing (in the bottom graph and noted as picture 4) real-time PCR quantification of B. burgdorferi on the tick DNA preps isolated using shredder-PCT, wherein Borrelia burgdorferi infections estimation for the tick DNA preps isolated using shredder-PCT, (real-time PCR amplification was performed using the same set of primers and probe as those in the standard curves, picture 3, the graphs shows B. Burgdorferi DNA spiked and non-spiked;

FIG. 9 is a bar graph showing the effective DNA recovery from spinach samples using only PCT, shredder in accordance with some embodiments of the invention, and coupled shredder/PCT techniques in accordance with some embodiments of the invention;

FIG. 10 is a copy of a photograph showing the relative level of recovered DNA using agarose gel electrophoresis as extracted from spinach leaf utilizing a Bead Beater technique, shredder and PCT coupled technique, and PCT alone;

FIG. 11 shows a graph and a copy of a photograph of recovered DNA from spinach leaf at various shredding conditions in accordance with some embodiments of the invention;

FIG. 12 is a graph showing the recovered protein levels from cardiac muscle using the shredding and PCT techniques, in accordance with some embodiments of the invention;

FIG. 13 shows the identification of Borrelia burgdorferi DNA from tick DNA Ixodes scapularis;

FIG. 14A is a copy of a photograph showing the total protein from cardiac muscle as visualized on an SDS-PAGE gel stained with coomasie blue;

FIG. 14B is a graph showing protein quantification by Bradford assay (the total protein was extracted in PBS (n=9 per group) or ProteoSolve-IEF reagent (n=6 per group);

FIGS. 15A and 15B are copies of photographs of a PCT shredder apparatus with a driver and holder (FIG. 15A) and a shredder PULSE tube (FIG. 15B);

FIGS. 16A to 16D show a graph and copies of photographs of comparative protein yield following PCT or bead beating;

FIGS. 17A to 17D are copies of photographs showing nematode samples;

FIG. 18 is a copy of a photograph showing DNA visualization by gel electrophoresis;

FIG. 19 is a graph showing a comparison of DNA recover from spinach lead utilizing various techniques; and

FIG. 20 is a graph showing a comparison of DNA Recovery from spinach leaf by Bead Beating, PCT/shredding, and PCT.

### DETAILED DESCRIPTION

The use of shredding mechanical disruption processes of the present invention can allow the use of less aggressive buffers.

Simple mechanical grinding processes, such as in a mortar and pestle, may be undesirable because of the likelihood of sample cross contamination, potential exposure of the lab workers to dangerous samples, and uncontrolled grinding force and duration. The use of a single disposable container which can be used from sample collection, to gentle mechanical disruption, to pressure cycling extraction can increase sampling accuracy and reproducibility, prevent contamination, and protect the user from sample exposure due to unconfined sample processing.

One or more embodiments of the systems and techniques of the present invention can utilize controlled compressive and rotating action under controlled time to achieve gentle rapid disruption of a sample's external structure as a preparatory processing procedure for pressure cycling techniques, such as those utilized in the systems and processes disclosed by Pressure Biosciences, Inc., South Easton, Mass., to gently separate, for example, cellular components of interest. The various aspects of the presently disclosed controlled compressive rotation can effect mechanical disruption that facilitates a highly flexible sample preparation process capable of being utilized on many different types of samples. Further, in particularly notable embodiments of the invention, the reproducibility and total sample extraction yield is typically enhanced by the synergistic action of pressure cycling techniques with the systems and techniques of the invention.

In a particular configuration, the systems and techniques of the present invention utilize controlled compressive and rotating forces and a timer to achieve high reproducibility during processing. The thrust and rotating action can be automatically generated, machine generated, or manually generated by a user with or without a feedback indicator that provides a quantitative representation of the amount, level, and/or duration of energy delivered or applied to one or more samples.

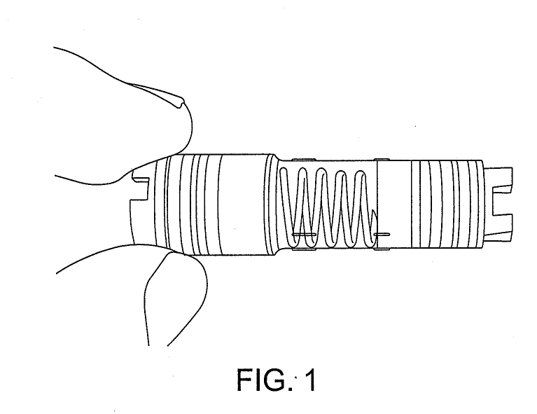

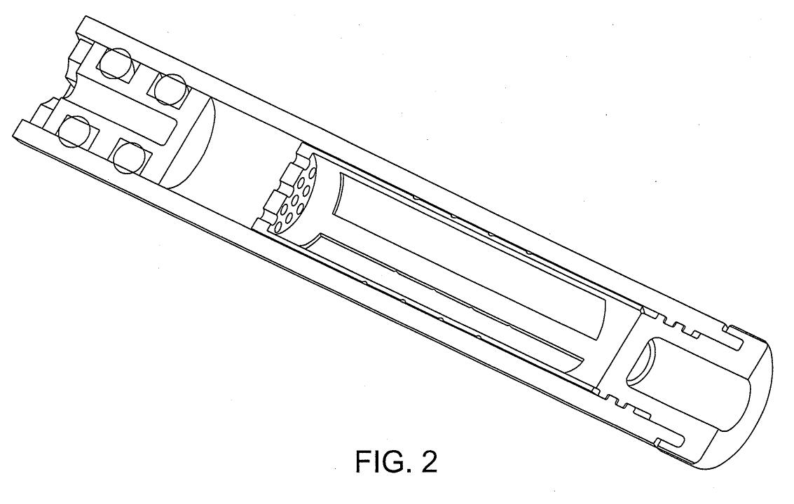

An alternative to or in conjunction with the above described shredder, gentle rotation processes, a spring or other elastic element inside a pulse tube to support a lysis disk as illustrated in FIG. 1. By doing this, ram positioning is no longer highly important to the pressure cycling process. The lysis disk is now capable of moving to avoid excessive force that can implode the tube. The spring loaded lysis disk can also allow the ram to be placed into contact with the lysis disk at the start without fear that the tube will implode. The stiffness of the spring or elastic element can be selected to be under the force that would damage tube due to overpressure. The elastic element can be designed so that rotation is resisted to facilitate shredding processes to gentle rotation approach to tear open tough enveloped samples. The elastic element can be a mechanical spring or flexible design features of a component. For example, the lysis disk can be supported by individual legs as illustrated in FIG. 2 that can bend when subjected to loading. By allowing both shredding by rotation and ram to lysis disk contact during high pressure cycling, the maximum extraction can be achieved with tough enveloped samples.

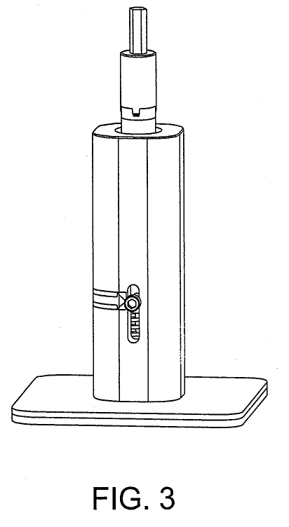

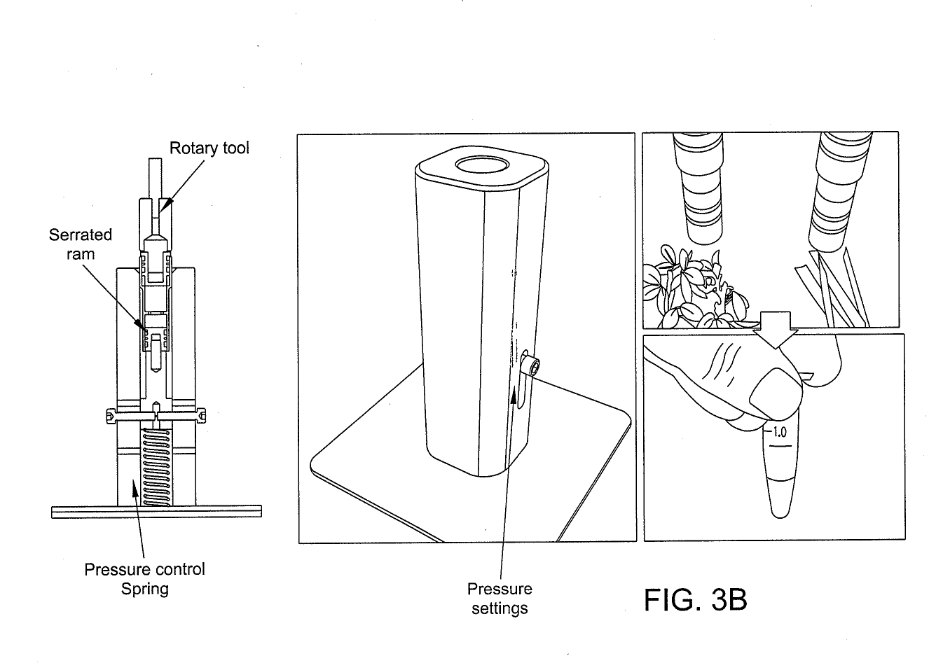

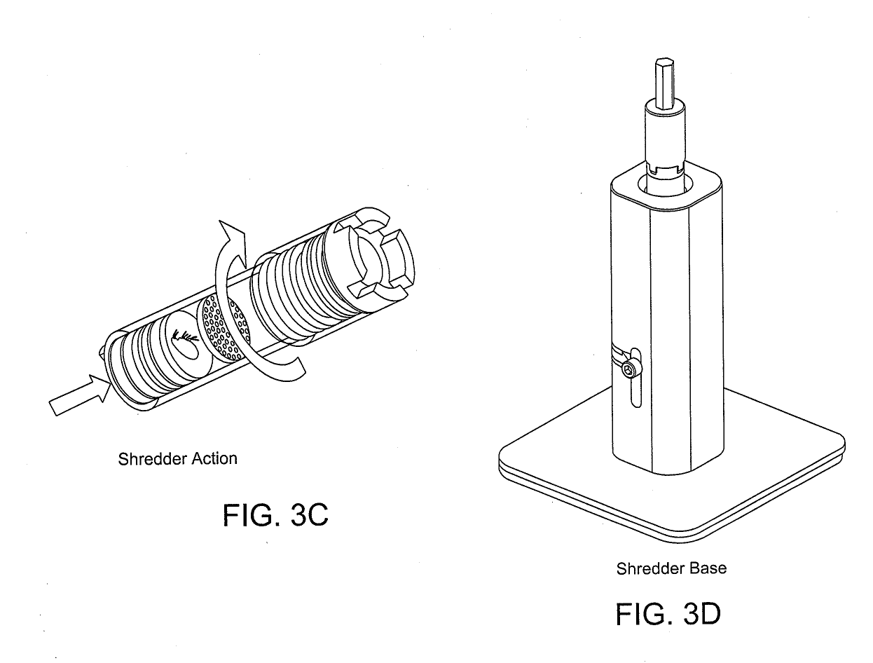

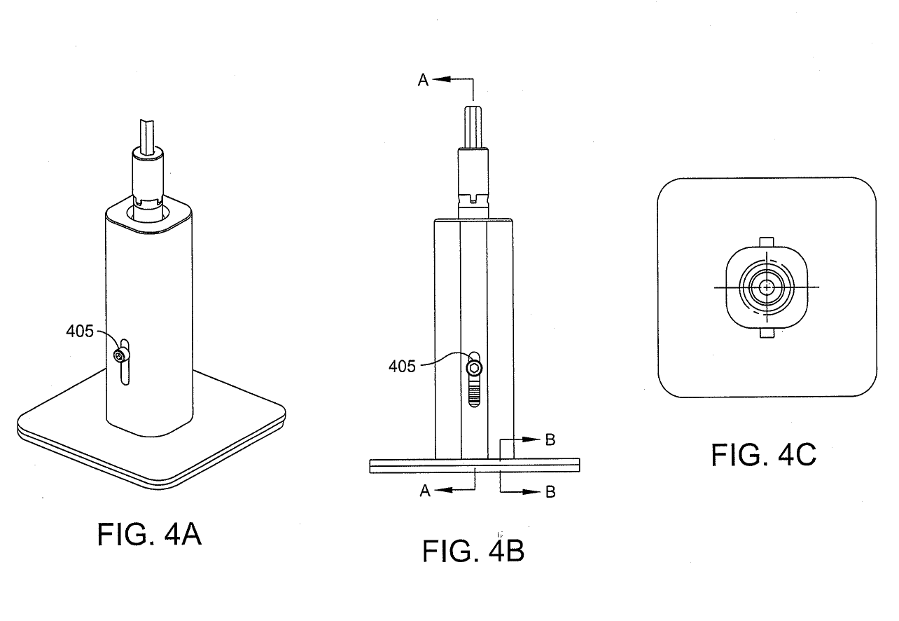

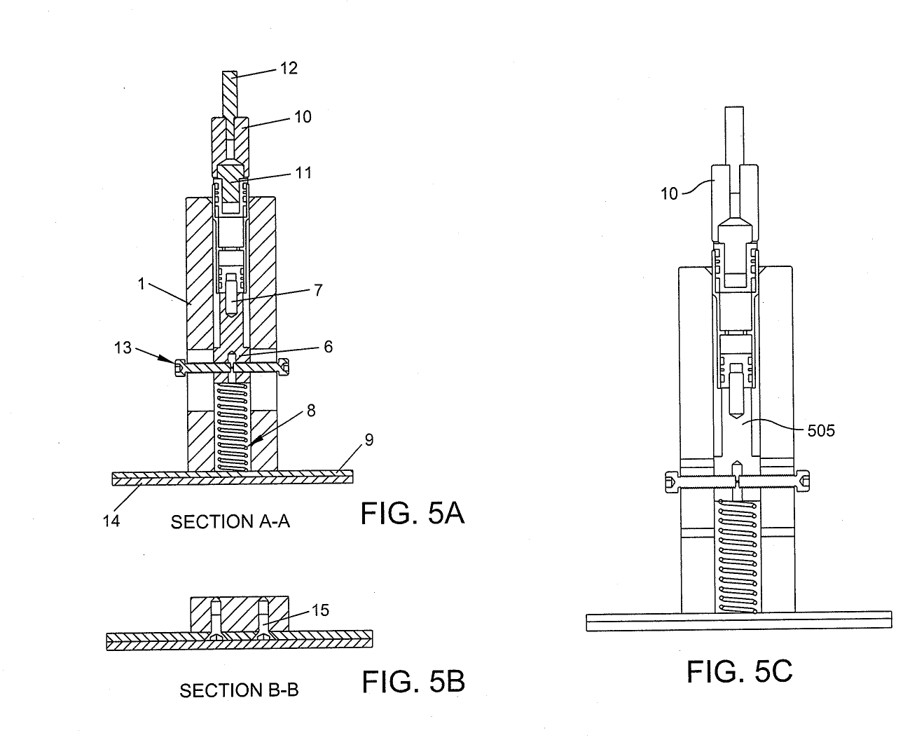

In an embodiment of the invention, the sample tube, which can be a tube as disclosed in by Schumacher in U.S. Patent Application Publication No. 2002/0197631, and which is incorporated herein by reference in its entirety for all purposes, is positioned on a spring-loaded non-rotating platform. The sample tube, illustrated in FIG. 1, can be disposed in an assembly, as illustrated in FIGS. 3A-3D and 4A-4C, or be operatively coupled to at least one indicator 405 that provides a user an indication or magnitude of one or more applied forces. The at least one indicator can provide at least one of a representation of a thrust force, compressive force, and a rotational or shear force applied on a sample or at least a portion of the sample tube. Based on the indicia, the user can adjust the magnitude of applied forces. For example, if the indicator provides a representation of applied forces that are below or above a target, preselected or predetermined level, the applied forces can be increased or decreased, respectively, to reduce any deviation between the target force and the actual applied force.

The indicator can have a plurality of indicia can represent a plurality of applied force levels. For example, the indicator can have a graded scale and a displaceable pointer that provides a plurality of relative position indications each of which corresponding to an applied force. The present invention, in at least one embodiment, can utilize different springs that provide a reactive force against the applied force, which can increase the operating range of the device. In alternative embodiments, a reactive force can be generated by opposing magnets, fixed weights, or elastic elements.

In accordance with still further embodiments, an automatic mode can be utilized wherein at least one of the applied thrust force, compressive force, and rotational force can be machine generated and controlled. In other embodiments, this invention can be automated, the thrust force can be machine controlled or generated by mechanical means such as an air cylinder or electromagnets. Multiple samples can be processed in an automatic device.

A simple handheld fixed rotational speed (e.g., rpm) motorized device, such as a cordless screwdriver can be utilized to generate rotational or shear forces. The rate or rotation and the viscosity of the sample can be determinant of the magnitude of the applied rotational force. For example, a rotation rate of no more than 250 rpm may advantageously avoid heating that can result in undesirable sample degradation.

After rotational and compressive or thrust processing, additional fluids can be added to the tube, and the sample tube can be inserted into a PCT BAROCYCLER pressure cycling device, from Pressure Biosciences, Inc., with the sample still inside. Some operational variations could involve the freezing of the sample prior to pre-processing. Other operational variations could involve the use of a pulse tube without a lysis disk wherein a ram of the tube is rotated against the cap of the pulse tube. The pulse tube ram or cap can be textured to help generate more sample tearing action. Alternatively, hard inert solid particles can be used in the tube in to facilitate sample tearing. For example, alumina abrasive particles can be added to the sample during any of one or more of the rotational and compressive processes.

A sample such as a whole insect is typically placed between a movable ram and a lysis disk of the sample tube or sample container, with or without a selected buffer solution. The sample tube is positioned between a non-rotating ram holder 505 and a rotating tube engagement tool 10. Tool 10 can be attached to a handheld motorized device (not shown) to rotate the tube in relation to the ram. The sample tube is contained within the body of the device or assembly. The assembly can be built internal to a sample preparatory system, such as the PCT BAROCYCLER™ pressure cycling device, or be erected as a self standing configuration, as shown.

An arrow or other mark on the body of the device can serve as indicia to indicate the magnitude of the force being applied to the sample or to the sample tube.

Multiple positions can allow the simultaneous processing of a plurality of tubes.

In yet other embodiment, the sample can be frozen in the sample tube, such as a PULSE™ tube, from Pressure Biosciences, Inc., prior to shredding processes, which can facilitate the mechanical disruption and allow formed ice crystals to interact with the sample components during the controlled disruption by rotation.

In yet other embodiments, the shredder may provide sufficient disruption on certain sample types and PCT processing is not needed.

### Example 1

### Comparison of Shredder of the Present Invention and BIOMASHER™ (from Nippi, Inc.) in Extracting Protein from Pine Needles with a Physiological Buffer or ProteoSOLVE™ IEF Reagent

Pine needles were coarsely cut to about 4-5 mm lengths within one hour of harvesting. Either 50 or 200 mg was weighed into tared PULSE Tubes or BioMasher™ inserts. Samples were processed in duplicate, either in KPO₄ buffer or the ProteoSOLVE IEF Reagent with 100 mM DTT.

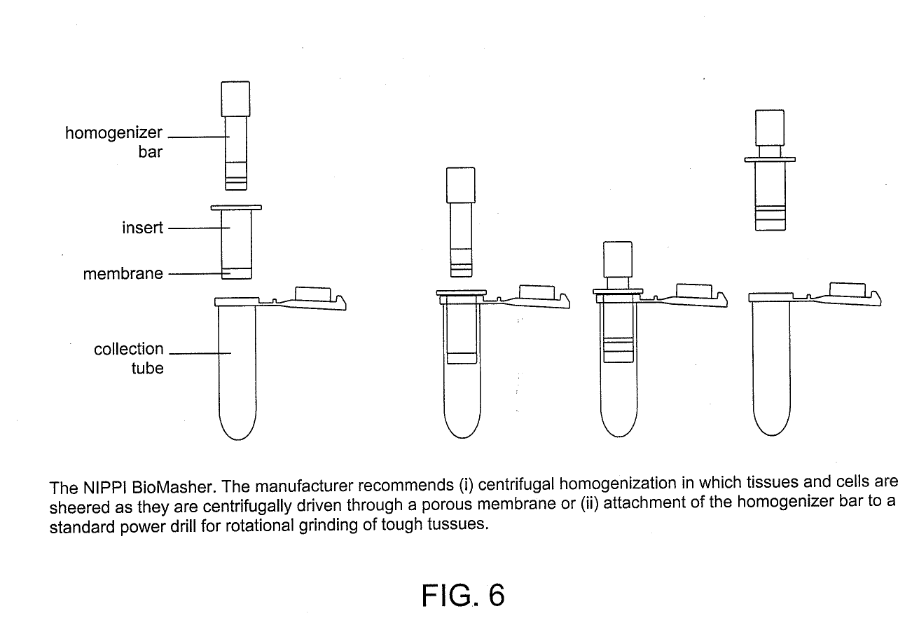

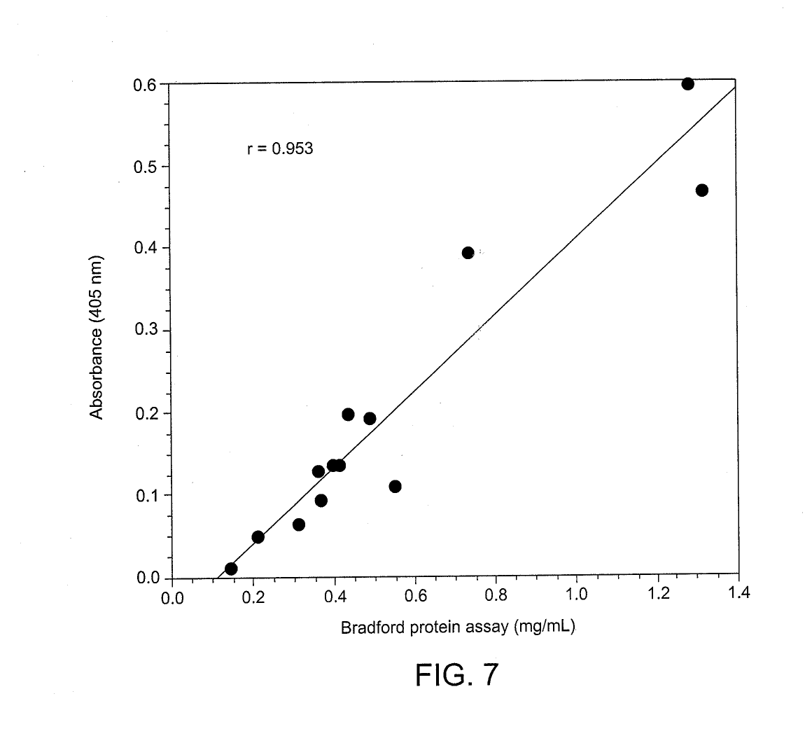

As illustrated in FIG. 6, for the BioMasher™ centrifugal method, the assemblies were centrifuged at 14,000 for 20 seconds with homogenizer bar positioned according to the manufacturer's instructions. BioMasher™ inserts were 80-140 μm pore size. The inserts were washed twice, each time with 700 μL followed by centrifugation. Initial homogenates and washes were pooled. Final sample volume was 1400 μL. For the Biomasher™ rotational grinding method, the homogenizer bar was connected to a standard power drill according to the manufacturer's instructions. Samples were ground for 30 seconds, followed by centrifugation at 14,000 for 20 seconds. The inserts were washed twice, each time with 700 μL followed by centrifugation. Initial homogenates and washes were pooled. Final sample volume was 1400 μL.

For the Shredder, the samples were ground for 30 seconds followed by the addition of 1400 μL KPO₄ buffer or ProteoSOLVE IEF reagent with 100 mM DTT. All PCT processes were performed for 40×10 second cycles at 35,000 psi. Supernatants were clarified by centrifugation at 14,000 RCF followed by further clarification. Protein concentration of supernatants was estimated using the Bradford reagent. Relative tannin concentrations were estimated spectrophotometrically at 405 nm.

Tables 1, 2, 3, and 4 show the increased yield of protein in the PCT Shredder process as compared to purely mechanical disruption process (BIOMASHER™) in ProteoSOLVE™ IEF reagent and physiological buffer solution. While the Shredder of the present invention provides achieve a high level of protein yield, the combination of PCT processes with the Shredder of the invention can achieve higher yields. Increased yield of protein could allow the detection on low abundance proteins important to many researchers.

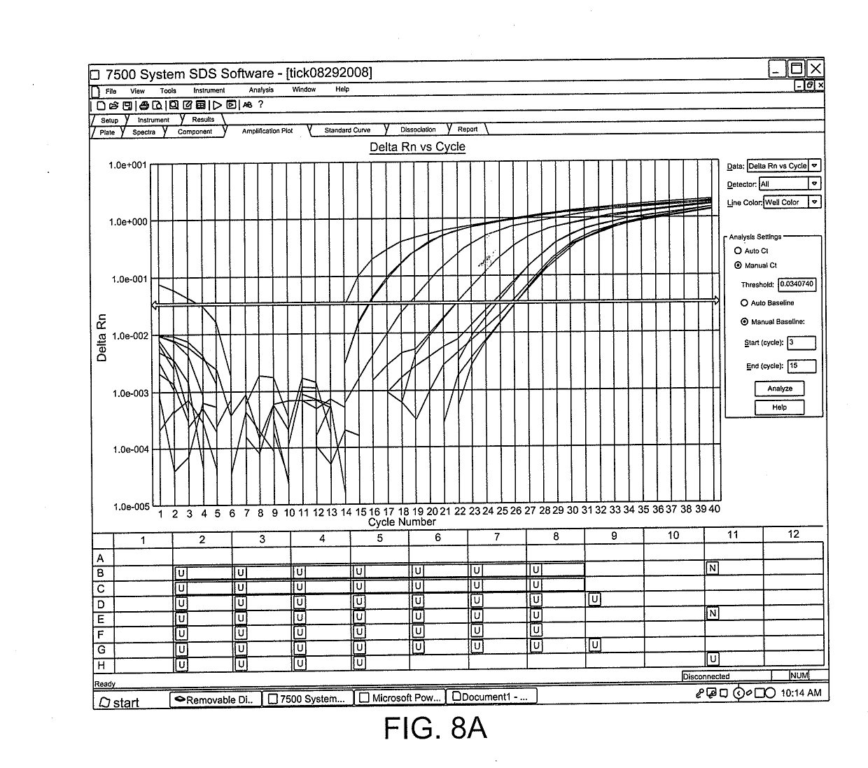

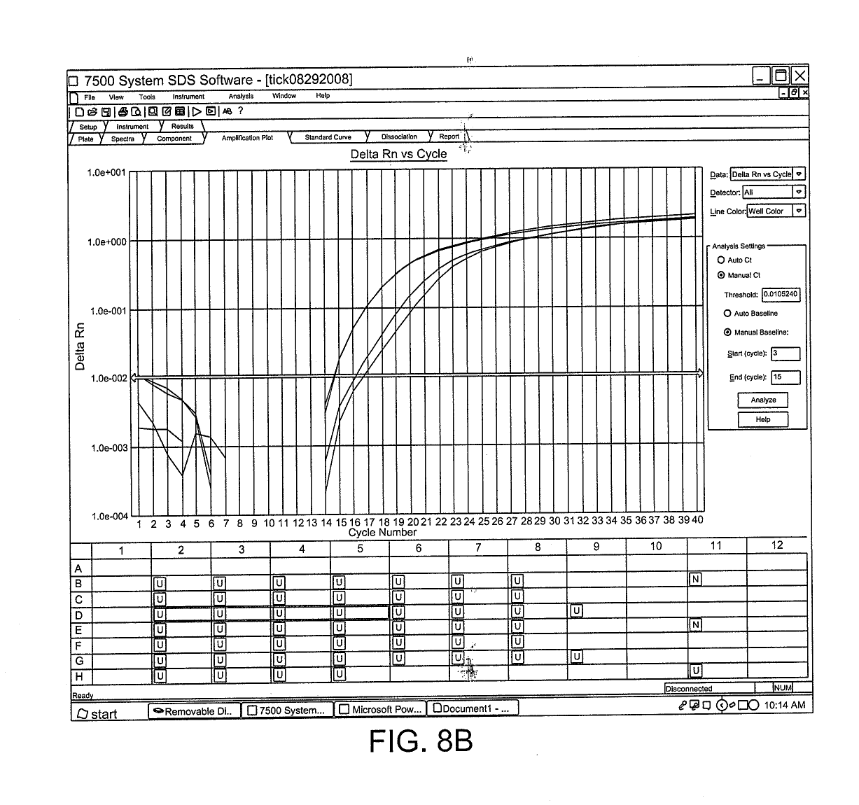

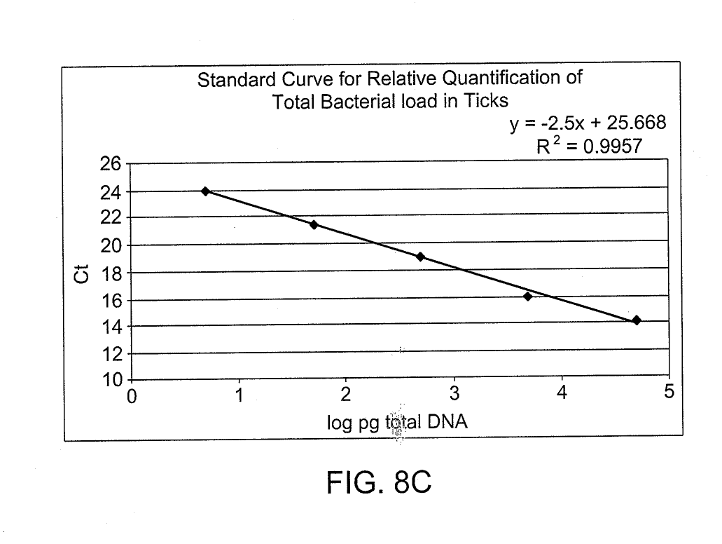

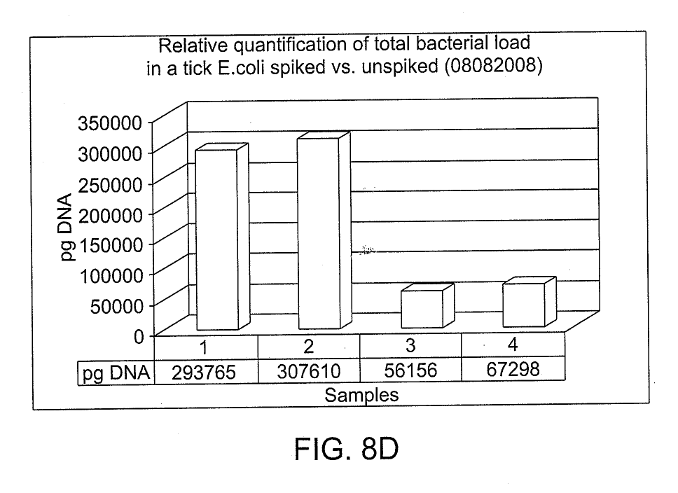

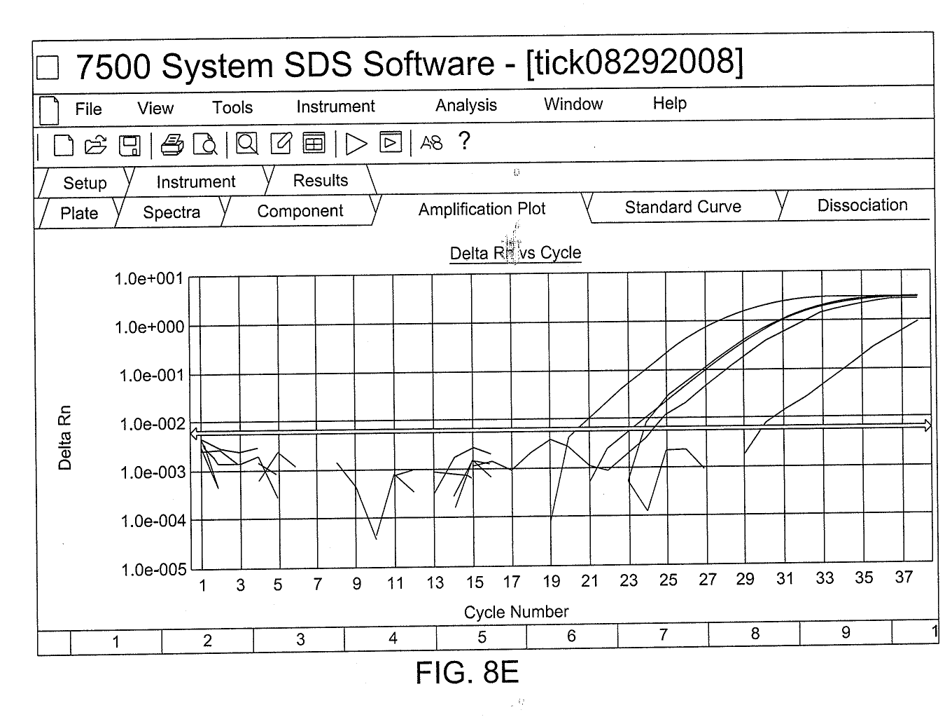

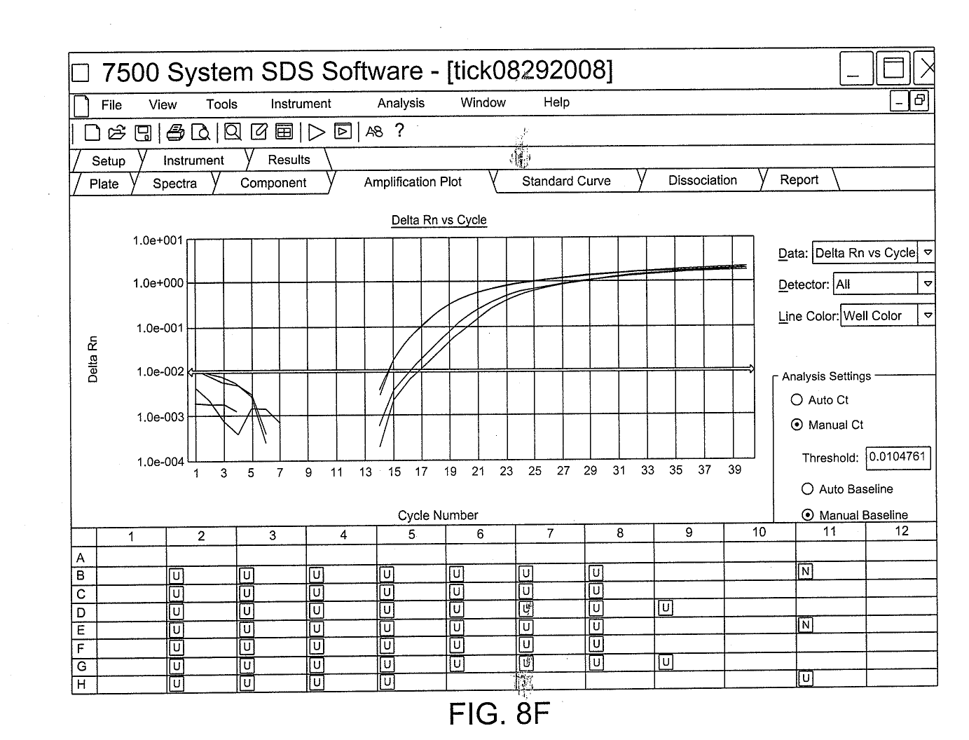

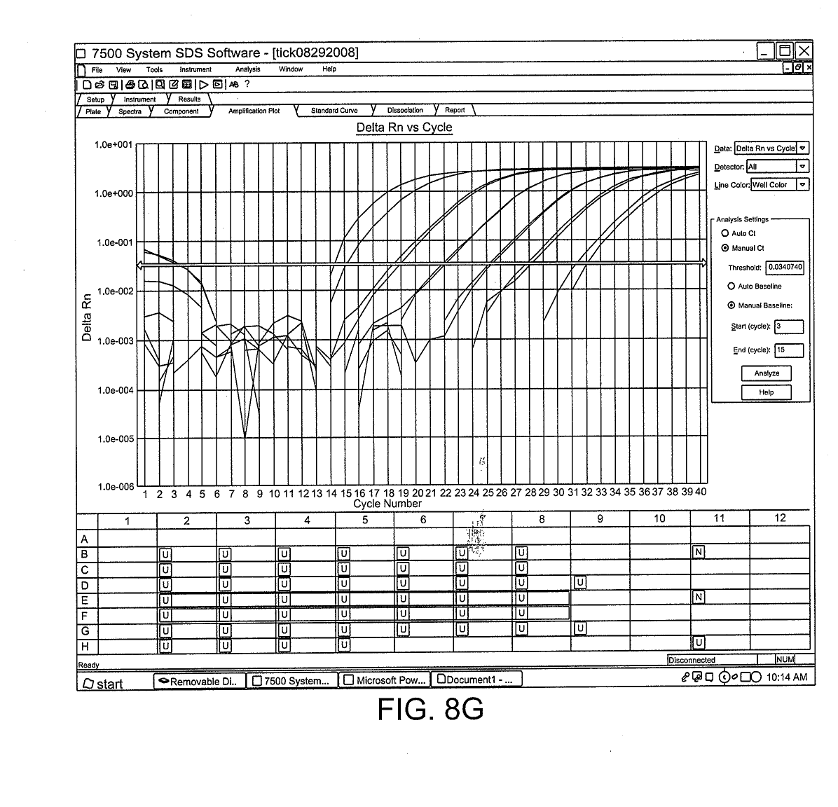

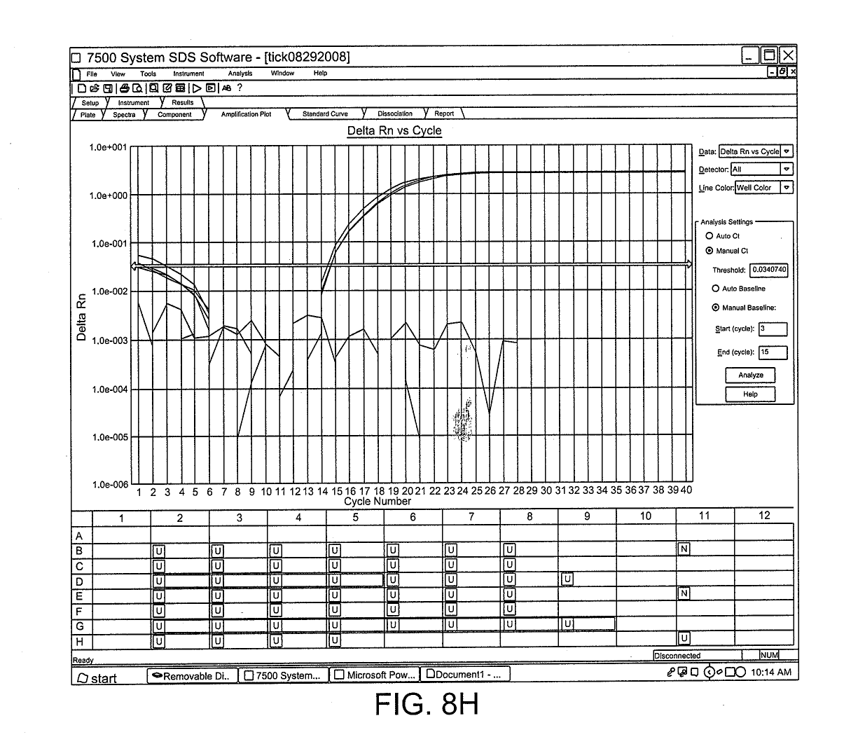

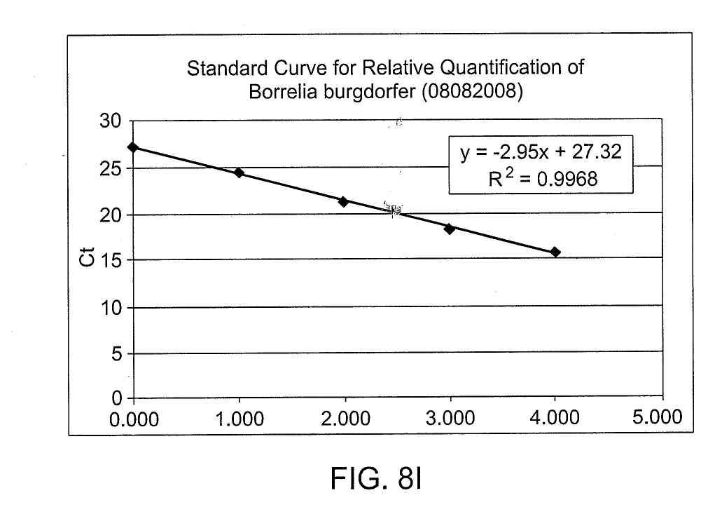

FIG. 8D shows that combined shredder and PCT tick extracts was strongly amplified to identify characteristics genes. Non shredder PCT samples were not successful in achieving amplification.

The results show that the highest protein yields from 50 or 200 mg pine needle samples were obtained from ProteoSOLVE IEF Reagent using the Shredder followed by PCT. The BioMasher insert has a volume capacity of 600 uL making processing of samples much larger than 50 mg cumbersome.

Rotational grinding of 200 mg samples occasionally damaged the porous membrane, resulting in flow through of sample.

Centrifugal homogenization in the BioMasher was ineffective for extracting proteins from pine needles. Rotational grinding increased protein yields 47% and 336% in KPO₄ buffer and ProteoSOLVE IEF reagent, respectively.

Rotational grinding in the BioMasher did not increase protein yields when sample size was increased from 50 to 200 mg. Increase over negative control was 336% and 119%, respectively, suggesting limitations of sample size.

Rotational grinding in the Shredder was more effective than the BioMasher for 200 mg samples, likely due to the larger grinding surfaces.

TABLE 1
50 mg pine needles in PROTEOSOLVE ™ IEF reagent

| | Actual | | |
|---|---|---|---|
| | Sample Mass | Protein Assay | Tannins |
| Description | (mg) | (mg/mL) | (Abs 405 nm) |
| BIOMASHER ™, | 48 | 0.148 | 0.012 |
| centrifugal | | | |
| BIOMASHER ™, | 51.5 ± 1.5 | 0.497 ± 0.081 | 0.154 ± 0.062 |
| rotational grinding | | | |
| Shredder, no pressure | — | — | — |
| control | | | |
| Shredder, PCT | 54.5 ± 0.5 | 0.275 ± 0.051 | 0.057 ± 0.010 |

TABLE 2
200 mg pine needles in PROTEOSOLVE ™ IEF reagent

| | Actual | | |
|---|---|---|---|
| | Sample Mass | Protein Assay | Tannins |
| Description | (mg) | (mg/mL) | (Abs 405 nm) |
| BIOMASHER ™, | 199 | 0.368 | 0.093 |
| centrifugal | | | |
| BIOMASHER ™, | 202 ± 1.0 | 0.437 ± 0.048 | 0.163 ± 0.041 |
| rotational grinding | | | |
| Shredder, no pressure | 198 | 0.737 | 0.391 |
| control | | | |
| Shredder, PCT | 200.5 ± 4.5 | 1.302 ± 0.021 | 0.530 ± 0.092 |

TABLE 3
50 mg pine needles in 50 mM KPO₄ buffer, ph 7.4

| | Actual | | |
|---|---|---|---|
| | Sample Mass | Protein Assay | Tannins |
| Description | (mg) | (mg/mL) | (Abs 405 nm) |
| BIOMASHER ™, | 49 | 0.074 | 0.230 |
| centrifugal | | | |
| BIOMASHER ™, | 49.5 ± 0.5 | 0.109 ± 0.003 | 0.186 ± 0.044 |
| rotational grinding | | | |
| Shredder, no pressure | — | 0.077 ± 0.015 | 0.171 |
| control | | | |
| Shredder, PCT | 51.5 ± 2.5 | 0.081 ± 0.002 | 0.129 ± 0.008 |

TABLE 4
200 mg pine needles in 50 mM KPO₄ buffer, ph 7.4

| | Actual | | |
|---|---|---|---|
| | Sample Mass | Protein Assay | Tannins |
| Description | (mg) | (mg/mL) | (Abs 405 nm) |
| BIOMASHER ™, | 203 | 0.098 ± 0.015 | 0.115 |
| centrifugal | | | |
| BIOMASHER ™, | 199 ± 5.0 | 0.287 ± 0.171 | 0.635 ± 0.110 |
| rotational grinding | | | |
| Shredder, no pressure | 195 | 0.077 ± 0.015 | 0.171 |
| control | | | |
| Shredder, PCT | 203.5 ± 3.5 | 0.103 ± 0.035 | 0.401 ± 0.155 |

### Example 2

### Increased Protein Yields from Coniferous Plants Using the PCT Shredder™ and Pressure Cycling Technology (PCT)

The plant proteome provides the opportunity to monitor post-translational response to environmental influences such as pollution, insect infestation, or plant diseases. Comprehensive proteomic analyses require reliable extraction methods that isolate proteins reproducibly and without bias. Sample preparation of plant tissues is particularly challenging due to the nature of cell walls, which make it difficult to quantitatively extract analytes, the relatively low cellular content of proteins in some plant tissues, or the abundance of lignin, tannin, and other polyphenols that can interfere with protein analyses. The extraction of proteins from pine needles and other coniferous tissues is particularly challenging, and may be further complicated in these species by their high content of terpene resins. Here a system for the efficient extraction of proteins from two conifers, Pinus strobus (Eastern White Pine) and Thuja standishii (Japanese Arborvitae) is described.

Initial disruption of plant tissue with the PCT Shredder followed by protein extraction using pressure cycling technology (PCT) is carried out in the presence of various extraction buffers in the same processing container (Shredder PULSE Tube). This method of extraction is safe, convenient and efficient. In combination, the PCT Shredder and PCT extracted up to 2-3 times more protein from pine needles than the BioMasher™ centrifugal homogenization device (See Table 5). Data also show that nondenaturing and strong denaturing extraction buffers can effectively be used in combination with the PCT Shredder and PCT to extract proteins from both P. strobus and T. shandishii for analysis.

Pressure Cycling Technology (PCT)

In the Pressure Cycling Technology Sample Preparation System (PCT SPS) hydrostatic pressure was rapidly cycled between ambient and ultra high levels (35,000 psi) to control biomolecular interactions. High hydrostatic pressure acts preferentially on the compressible components of the sample, such as cell membranes, resulting in cell lysis and the release of intracellular contents. The PCT SPS can be used to disrupt plant and animal tissues, cells, cellular structures and microbes to extract nucleic acids, proteins and lipids. The system is comprised of a small, semi-automated bench-top instrument (Barocycler NEP3229 or the NEP2320) used in combination with single-use sample processing containers (PULSE Tubes). PCT in the presence of suitable extraction reagents, such as nondenaturing or strong denaturing buffers results in isolation of proteins for analysis.

The PCT Shredder

The PCT Shredder was designed to physically disrupt and enhance extraction of tough, fibrous and other difficult-to-disrupt biological materials such as certain plant and animal tissues. The PCT Shredder was used to rapidly grind the sample directly in a specially designed Shredder PULSE Tube to increase the tissue surface area and to improve cell lysis prior to treatment by PCT for extraction of nucleic acids, proteins, lipids and other cellular contents. Since shredding and PCT were performed in the same tube, loss of sample or the likelihood of cross contamination was significantly reduced as compared to other processing methods.

Materials and Methods

Needles from P. strobus and leaves from T. standishii were harvested and processed within one hour of collection. The plant tissue were then coarsely chopped to 2-3 mm length and weighed into tared Shredder PULSE Tubes or FT500 PULSE Tubes in 50, 200, or 350 mg aliquots to which 1350 μL of nondenaturing buffer (ProteoSolve NATIVE or NATIVE Plus) or strongly denaturing buffer (ProteoSolve CE PrEP Kit or ProteoSolve SB Kit) was added. All reagents were supplemented with protease inhibitors (Sigma-Aldrich, St. Louis, Mo.). PCT was performed with and without using the PCT Shredder to demonstrate the efficacy of extraction by each method, as well as to demonstrate the cumulative effect of using chemistry, the PCT Shredder and PCT in combination.

For comparison, 50 or 200 mg of conifer tissue was processed using a BioMasher centrifugal homogenizer (Cartagen Molecular, San Carlos, Calif.) by grinding against the porous polyethylene membrane of the BioMasher insert with the homogenizer bar. Two separate aliquots of 700 uL of buffer were added and the combined homogenate was collected by centrifugation. Coniferous biomasses larger than 200 mg could not be accommodated in the small insert of the BioMasher™.

Results and Discussions

The PCT Shredder yielded nearly twice the protein from 200 mg of P. strobus needles than the BioMasher centrifugal homogenization device (Table 5) and three times the protein than the BioMasher when followed by PCT for 40 cycles at 35,000 maximum pressure. From 50 mg samples, the PCT Shredder and the BioMasher yielded similar amounts of protein suggesting that the BioMasher™ was only effective for processing relatively small amounts of sample.

TABLE 5
Comparison of PCT Shredder and the BioMasher centrifugal
homogenizer for the extraction of protein from pine needles

| | actual sample | protein | tannins |
|---|---|---|---|
| description | mass (mg) | (mg/mL) | (405 nm) |
| BioMasher | 202.0 ± 1.0 | 0.437 ± 0.048 | 0.163 ± 0.041 |
| PCT Shredder only | 198 | 0.737 | 0.391 |
| PCT Shredder plus | 200.5 ± 4.5 | 1.302 ± 0.021 | 0.530 ± 0.092 |
| PCT | | | |

Proteins extracted from pine needles in Reagent D from the ProetoSOLVE SB Kit.

In other experiments, the effectiveness of various buffers in combination with the PCT Shredder and PCT were evaluated. ProteoSolve NATIVE and NATIVE Plus buffers were designed to extract proteins under relatively mild conditions. These buffers are required when preservation of the native conformation and biological activity of proteins prohibits the use of chaotropes or detergents. ProteoSolve NATIVE Plus contains a mild nondenaturing surfactant to increase the solubility of hydrophobic protein; its use resulted in a 56% increase in protein yields from the more resinous T. standishii compared to the other buffers evaluated (See Table 6).

TABLE 6
Comparative protein yields from T. standishii leaves using the
PCT Shredder and nondenaturing or strongly denaturing reagents.

| | extraction | protein assay | tannins |
|---|---|---|---|
| method | reagent | (mg/mL) | (405 nm) |
| PCT Shredder only | ProteoSolve | 0.133 ± 0.043 | 0.843 |
| | NATIVE | | |
| PCT only | | 0.270 ± 0.088 | 0.505 |
| PCT Shredder plus | | 0.410 ± 0.029 | 1.147 ± 0.228 |
| PCT | | | |
| PCT Shredder only | NATIVE Plus | 0.452 ± 0.048 | 1.153 |
| PCT only | | 0.369 | 0.386 |
| PCT Shredder plus | | 0.640 ± 0.072 | 1.194 ± 0.333 |
| PCT | | | |
| PCT Shredder only | ProteoSolve CE | 1.657 ± 0.302 | 0.814 |
| PCT only | | 1.855 ± 0.041 | 1.178 |
| PCT Shredder plus | | 2.456 ± 0.150 | 2.111 ± 0.166 |
| PCT | | | |

From 350.4±0.7 mg T. Standishii leaves.

The use of chaotropes, detergents and reducing agents effectively dissociates non-covalent protein interactions and covalent S—S linkages resulting in significantly higher protein recoveries. The ProteoSolve CE Reagent from the CE PrEP Kit was specifically designed for maximal protein yields from recalcitrant samples in applications where the preservation of biological activity is not required. ProteoSolve CE yielded an order of magnitude more protein from pine needles than the milder, nondenaturing buffers (See Table 7).

Conclusions

The synergy between mechanical disruption of the PCT Shredder and the use of high hydrostatic pressure is an effective method to extract proteins from coniferous needles and leaves from P. strobus and T. standishii. Comparisons show that the PCT Shredder used in combination with PCT yielded more total protein than either method individually. Relatively high protein yields were obtained under nondenaturing conditions using the NATIVE and NATIVE Plus buffers, which is desirable to preserve protein interactions and activity. However, even higher protein yields may be obtained using reagents such as ProteoSolve CE, although biological activity maybe lost, since chaotropes reduce many proteins to their primary structure.

TABLE 7
Comparative protein yields from P. strobas needles using the
PCT Shredder and nondenaturing or strongly denaturing reagents.

| | extraction | protein assay | tannins |
|---|---|---|---|
| method | reagent | (mg/mL) | (405 nm) |
| PCT Shredder only | ProteoSolve | 0.366 ± 0.007 | 0.121 |
| | NATIVE | | |
| PCT only | | 0.583 ± 0.068 | 0.162 |
| PCT Shredder plus | | 0.896 ± 0.008 | 0.215 ± 0.007 |
| PCT | | | |
| PCT Shredder only | NATIVE Plus | 0.270 ± 0.012 | 0.165 |
| PCT only | | 0.544 ± 0.018 | 0.204 |
| PCT Shredder plus | | 0.603 ± 0.021 | 0.291 ± 0.019 |
| PCT | | | |
| PCT Shredder only | ProteoSolve CE | 5.085 ± 0.224 | 0.271 |
| PCT only | | 3.712 ± 0.330 | 1.087 |
| PCT Shredder plus | | 5.461 ± 0.328 | 1.379 ± 0.066 |
| PCT | | | |

From 350.0±1.0 mg pine needles.

### Example 3

### Tick Borrelia and HGE Gene Expression Analysis on DNA Preps Isolated Using Shredder with PCT: Standard Curve and Total Bacteria Lyses

Basic Methodology for tick DNA extraction involved the following steps.

The tick samples were soaked in Tris buffer for 1 hour before PCT. One tick was loaded into the ram end and shredded by hand, followed by PCT treatment for 60 cycles at 56 C in protease K. The tubes were placed in boiling water and boiled for 10 min then unloaded. CTAB buffer was added up to final concentration of 2% and allowed to incubate at 65 C for 20 min. Phenol-chloroform purification was performed. The final volume of 100 ul was saved at −20 C.

Real-time PCR were performed. Two standard curves were designed for relative quantitation of Borrelia and total bacteria DNA. Doing so, Borrelia DNA and E. coli DNA from ATCC was series diluted. Borrelia 23S rRNA gene and bacterial 16SrDBA gene were amplified.

An XY plot with the log DNA input amount vs. Ct for graph, linear regression and R-squared value are illustrated. The DNA amount in the sample preps was calculated by using the standard regression equations and to provide relative quantitation.

FIGS. 8A-8D show the observations and results. In particular, FIG. 8D shows that the combining the shredder technique of the invention with PCT techniques provided strongly amplified results that facilitate or identify characteristic genes which were heretofore not successfully amplified.

### Example 4

### DNA Extraction from Fresh Baby Spinach Leaves

Triplicate samples of fresh baby spinach (200 mg each) were disrupted by one of three methods: PCT alone (35,000 psi, 30 cycles), Shredder alone (20 seconds), or Shredder followed by PCT. All samples were processed in DNAzol DNA Reagent (Invitrogen) according to the manufacturer's instructions for DNA isolation from plant tissue. DNA recovery is expressed as μgDNA per mg of spinach.

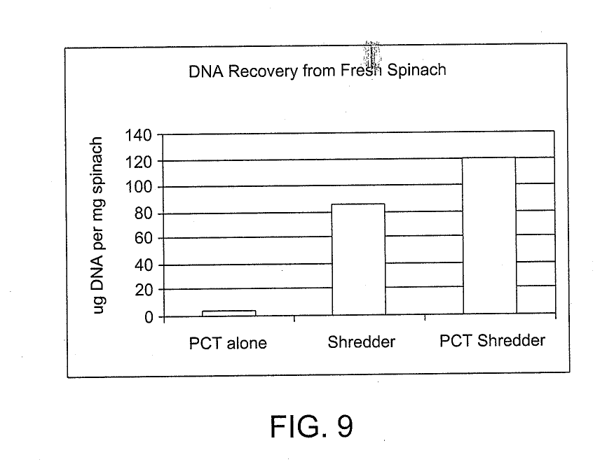

Tissue disruption by PCT alone resulted in a very low recovery of DNA (4.7+/−2 μg DNA per mg spinach, n=3). Tissue disruption with the Shredder of the present invention resulted in significant improvement in DNA recovery (86+/−42 μg DNA per mg spinach, n=3). Tissue disruption by The Shredder followed by PCT resulted in a further improvement is DNA recovery (121+/−51 μg DNA per mg spinach, n=3), as illustrated in FIG. 9.

### Example 5

### DNA Extraction from Fresh Baby Spinach Leaves compared to Bead Beater Processing

Triplicate samples of fresh baby spinach (200 mg each) were disrupted by one of three methods: PCT alone (35,000 psi, 30 cycles), Shredder (20 seconds) followed by PCT, or Bead beater (10 bursts of 10 seconds each at full power, samples chilled on ice between bursts). All samples were processed in DNAzol DNA Reagent (Invitrogen) according to the manufacturer's instructions for DNA isolation from plant tissue.

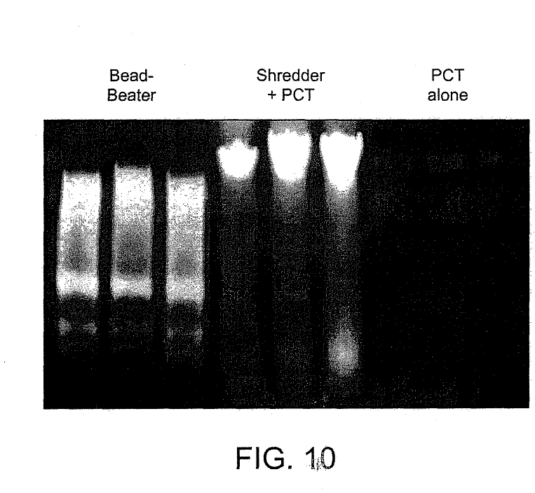

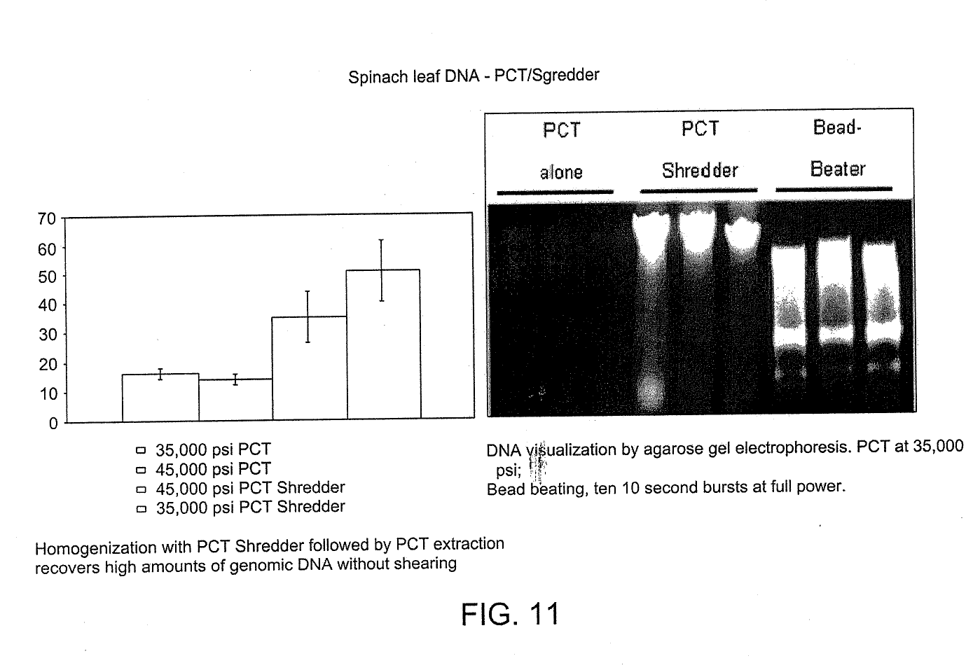

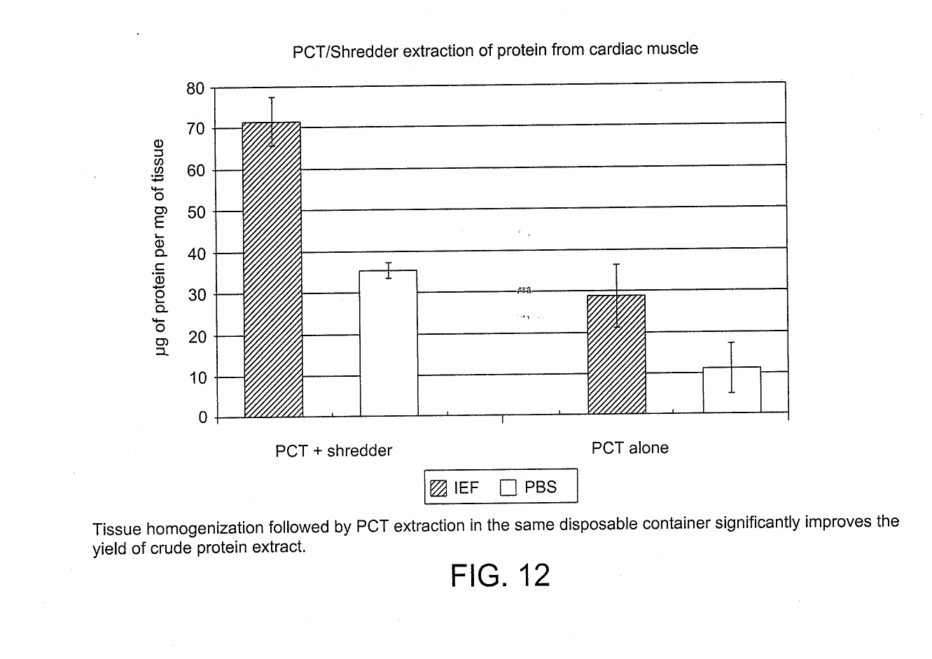

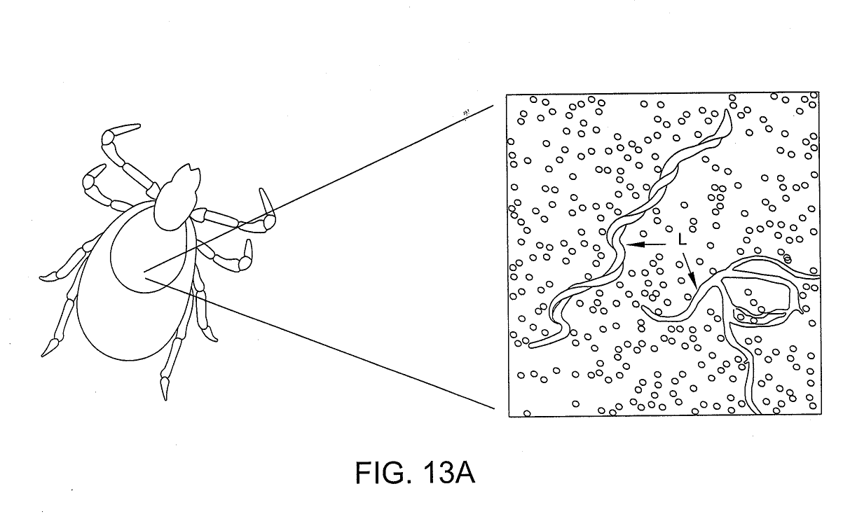

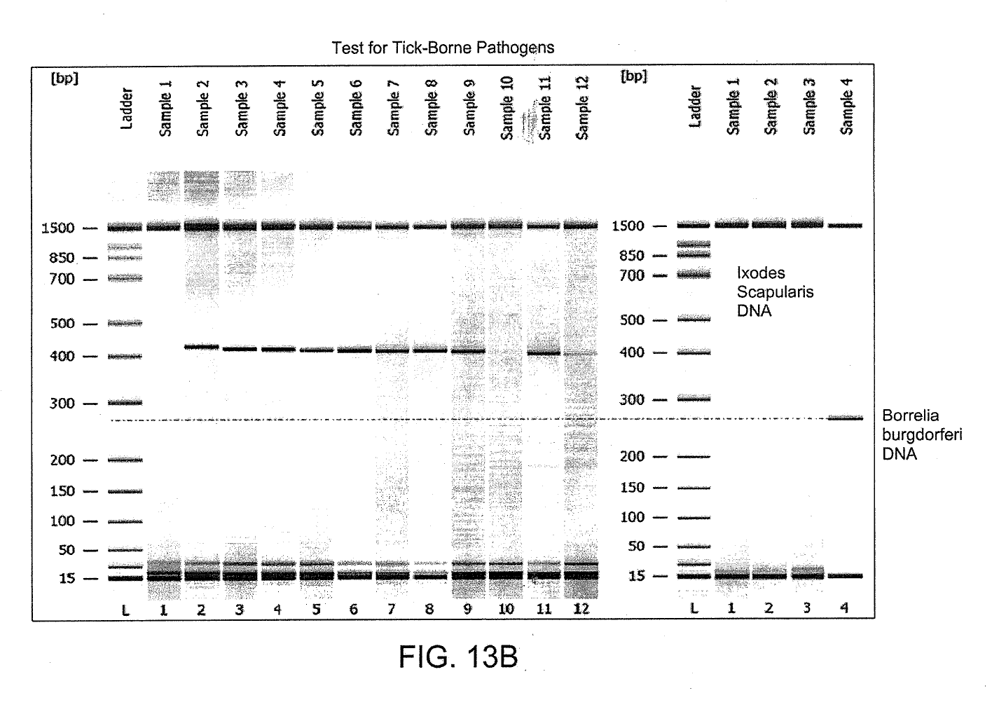

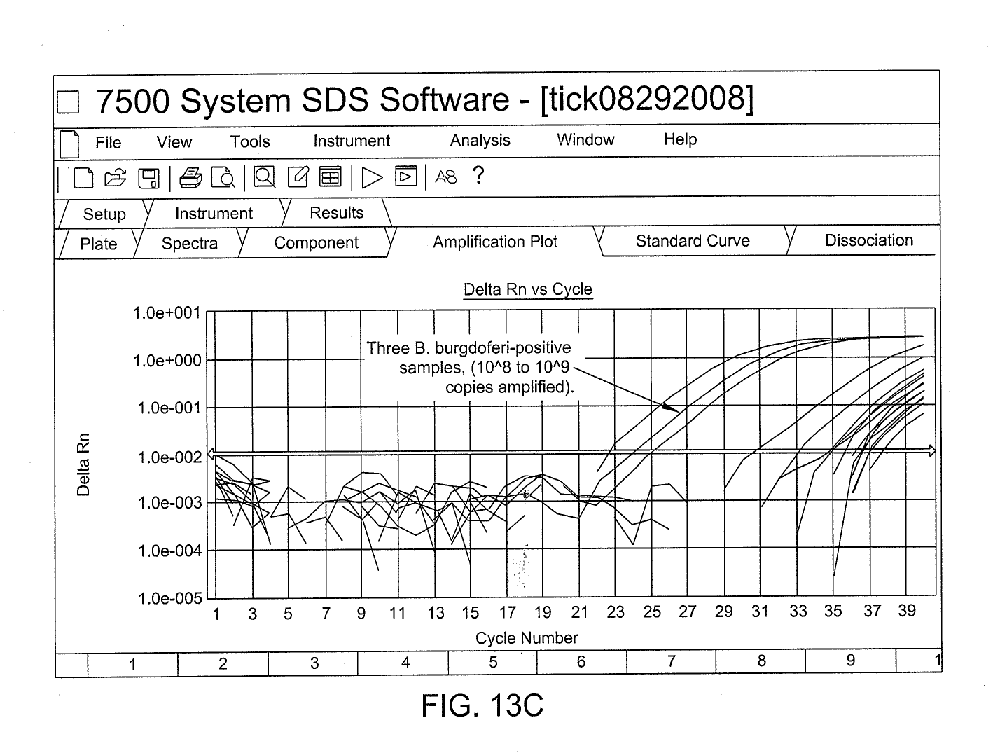

Tissue disruption by PCT alone resulted in a very low recovery of DNA (˜4.7 μg DNA per mg spinach). Tissue disruption by the shredder followed by PCT resulted in a significant improvement in DNA recovery (˜121 μg DNA per mg spinach). Tissue disruption with the Bead Beater technique resulted in higher DNA yield (268 μg DNA per mg spinach) however, as demonstrated by agarose gel electrophoresis, the recovered DNA was significantly sheared as illustrated in FIG. 10.

### Example 6

### RNA Extraction from Formalin-Fixed Paraffin Embedded (FFPE) Porcine Lymph Node Tissue by PCT Shredder with Proteinase K PrEP and ProteoSolve-SB

Whole or chopped blocks of deparaffinized tissue were placed into FT 500 PULSE Tubes with 0.8 ml ProteoSolve-SB Reagent A. The samples were disrupted with the Shredder for 20 seconds to increase sample surface area and improve access of the solvent to the sample. ProteoSolve-SB Reagent B was added (0.2-0.6 mL, to bring total reaction volume to 1.4 mL) and the samples were processed by PCT in the Barocycler NEP 3229 at 35,000 psi (240 MPa) for 30 cycles at 50° C.

After PCT, the samples were centrifuged and the RNA-containing insoluble material was separated from the lipid and solvent liquid phases. The solid pellet was dried in a vacuum evaporator for 5-10 minutes to reduce solvent carryover. The pellet was then dispersed in 0.7 mL of lysis buffer (Qiagen RNeasy™ buffer RLT) supplemented with 750 μg/mL Proteinase K and transferred to an FT 500 ND PULSE Tube. Digestion by Proteinase K was carried out at 20,000 psi (138 MPa) using 20-30 l minute pressure cycles at 50° C. Samples were decrosslinked by incubation at 80° C. for 15 minutes and then cooled to room temperature. Residual solid material was removed by centrifugation for 2 minutes at 800-900 g. The clarified supernatant was diluted with 3.5 volumes of absolute ethanol to enhance recovery of small RNA fragments. RNA was purified according to the RNeasy protocol (Qiagen). Table 8 shows the RNA Recovery results.

TABLE 8

| FFPE Tissue Mass | Total RNA Recovery |
|---|---|
| 100 mg | 3.6 μg |
| 100 mg | 4.8 μg |
| 100 mg | 2.6 μg |
| 100 mg | 8.0 μg |
| 100 mg | 6.0 μg |
| 100 mg | 1.8 μg |
| 100 mg | 3.8 μg |
| 100 mg | 3.3 μg |
| 100 mg | 4.6 μg |
| 100 mg | 0.4 μg |
| 100 mg | 0.9 μg |
| 100 mg | ND |
| 50 mg | 0.1 μg |
| 50 mg | 0.4 μg |
| 50 mg | 3.1 μg |
| 50 mg | 0.5 μg |
| 50 mg | ND |
| 50 mg | ND |

ND = No RNA detected, possible sample degradation prior to extraction

### Example 7

### Improved Protein Recovery from Muscle Tissue Using the PCT Shredder™ and Pressure Cycling Technology (PCT): Efficient Single Tube Sample Disruption and Extraction

Rapid, efficient and reproducible extraction of proteins from muscle is crucial for proteomic analysis and for studies of various conditions such as aging, hypertension, hypoxia and reperfusion-induced damage. Protein extraction from tough tissues such as cardiac and skeletal muscle generally requires extensive mechanical or chemical disruption of the same to adequately analyze its proteome. Mortar and pestle grinding, pulverization in liquid nitrogen or homogenization with a dounce or polytron homogenizer are some of the classical methods that can be used for the disruption of muscle tissue. However, these manual methods are inherently inconsistent, time consuming and potentially hazardous. Here we describe a system for efficient tissue disruption and protein extraction from bovine cardiac muscle, in a single sample processing tube, using the PCT Shredder and the Pressure Cycling Technology Sample Preparation System (PCT SPS). Mechanical tissue disruption with the Pct Shredder of the present invention provides rapid and convenient; and, when it is combined with the power of PCT, an efficient and reproducible method to prepare whole tissue lysates from tough samples such as cardiac and skeletal muscle.

Pressure Cycling Technology (PCT)

In the Pressure Cycling Technology Sample Preparation System (PCT SPS) hydrostatic pressure was rapidly cycled between ambient and ultra high levels (up to 45,000 psi) to control biomolecular interactions. High hydrostatic pressure acts preferentially on the compressible components of the sample, such cell membranes, resulting in cell lysis and the release of intracellular contents. The PCT SPS can be used to disrupt animal and plant tissues, cells, cellular structures and microbes to extract nucleic acids, proteins and lipids. The system is comprised of a small, semi-automated bench-top instrument (Barocycler NEP3229 or the NEP2320) used in combination with single-use sample processing containers (PULSE Tubes). PCT in the presence of suitable extraction reagents, such as DNAzol®, results in isolation of intact DNA for genomic analysis and other applications.

The PCT Shredder

The PCT Shredder was designed to mechanically disrupt the tissue in order to enhance extraction of tough, fibrous and other difficult-to-disrupt biological materials such as certain animal and plant tissues. The PCT Shredder was used to rapidly grind the sample directly in a specially designed Shredder PULSE Tube to increase the tissue surface area and to improve cell lysis prior to treatment by PCT in order to extract nucleic acids, proteins, lipids and other cellular contents. Since shredding and PCT are done in the same tube, loss of sample or the likelihood of cross contamination is significantly reduced as compared to other processing methods.

Materials and Methods

Thawed bovine cardiac muscle tissue (100 mg per sample) was processed in a Shredder PULSE Tube using the PCT Shredder as described in the User Manual (Pressure Biosciences, Inc.) in 0.4-0.5 mL of either Phosphate Buffered Saline (PBS) or ProteoSolve-IEF Reagent supplemented with 50 mM DTT. Subsequently, the cap to The Shredder PULSE Tube was removed and the sample volume was brought up to 1.4 mL with additional extraction buffer. The Shredder PULSE Tube was then re-capped with the PULSE Tube Cap and subjected to PCT under the following conditions: 35,000 psi held for 20 seconds, followed by atmospheric pressure held for 10-20 seconds; repeated for 20 cycles. All extractions were performed at ambient temperature. Following protein extraction in either PBS or in ProteoSolve-IEF, samples were centrifuged to pellet any residual solid debris and the clarified supernatant was used for protein quantification by Bradford protein assays (Bio-Rad). Protein assay results from replicate samples were averaged. Total protein from samples extracted in PBS was visualized by SDS-PAGE using 8-16% Criterion gels (Bio-Rad).

Results and Discussion

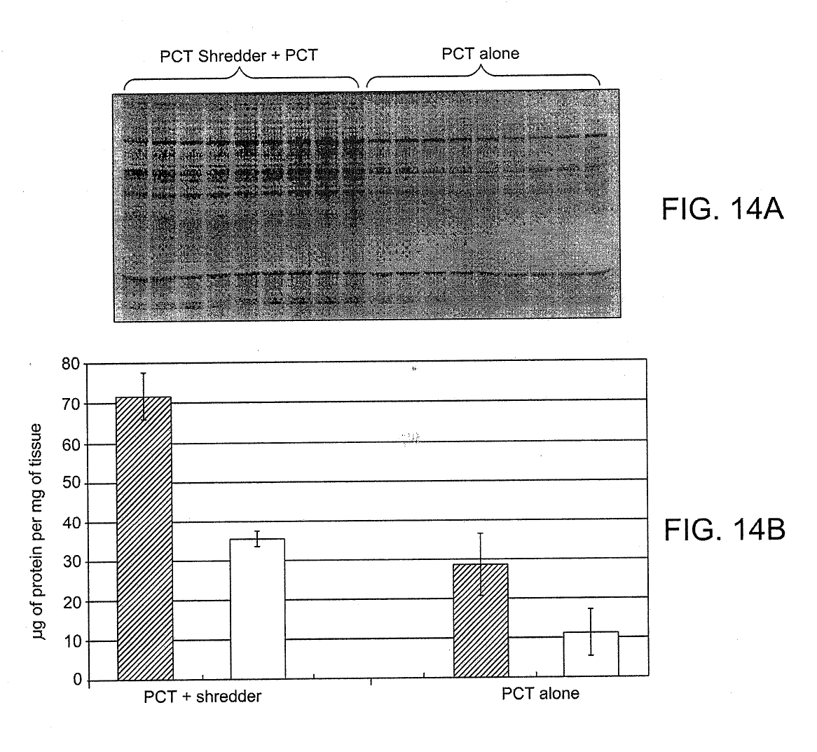

Protein recovery from samples processed by the PCT Shredder in PBS were 3.2-fold greater than samples processed by PCT alone (35.3+/−1.9 vs. 11.0+/−6.1 μg protein per mg of tissue), as illustrated in FIG. 14A. A similar improvement in protein recovery was observed in samples extracted in ProteoSolve-IEF reagent. The PCT Shredder increased total protein recovery in IEF buffer by 2.5-fold (71.7+/−5.9 vs. 28.7+/−7.6 μg proteins per mg of tissue). These data indicate that although buffer choice has a pronounced impact on protein recovery (FIG. 14B), use of the PCT Shredder in combination with PCT can improve protein extraction in various buffers and may allow the researcher to recover sufficient protein even in very mild buffer such as PBS. Data from both protein assays and SDS-PAGE analysis confirm that total protein recovery is significantly higher when muscle tissue is disrupted with the PCT Shredder prior to protein extraction by PCT. These results show that the preparation of crude total protein lysate from tough and fibrous tissue such as muscle is facilitated by the use of the PCT Shredder.

Conclusions

The combination of mechanical sample disruption by the PCT Shredder followed by extraction using pressure cycling technology (PCT) is an effective and safe method for isolation of protein from tough tissues such as cardiac and skeletal muscle. Rapid, efficient and reproducible extraction of proteins can be obtained for protomic analyses, even with very mild buffers such as PBS. In addition, since disruption and extraction are performed in the same container (Shredder PULSE Tube), sample recovery is enhanced, and the likelihood of cross-contamination is minimized.

### Example 8

### Pressure Enhanced Processing (PEP) Using the PCT Shredder and Pressure Cycling Technology (PCT) Maximizes Protein Yields from the Nematode Caenorhabditis Elegans Under Mild, Nondenaturing Conditions

The purpose of these experiments in this example was to develop an improved method to lyse the nematode Caenorhabditis elegans for proteomic studies. The tough exterior cuticle of Caenorhabditis elegans makes the nematode resilient to lysis. This resilience to lysis impedes proteomic analyses. Proteomic analyses are even more difficult when preservation of the native conformation and biological activity of proteins are desired, thus prohibiting the use of denaturing chaotropes or detergents to enhance lysis. In the course of our studies, it was determined that in physiological buffers, these nematodes are so resilient that they can even withstand hydrostatic pressure cycling technology (PCT) up to 20,000 psi with a 2.3% survival rate. However, by combining pressure and non-denaturing buffer with pre-processing using the present PCT Shredder, it was possible to achieve nearly total disruption of the nematodes and maximal protein yields. In these experiments, the nematodes were mixed with silicate (SIC) abrasive and frozen directly in a Shredder PULSE Tube™, a specialized container used in both the PCT Shredder and subsequent high pressure treatment in a Barocycler from Pressure Biosciences, Inc. To achieve optimum lysis, the frozen sample-abrasive mix was first ground with the PCT Shredder. The sample was then subjected to PCT. Damage to cuticles was evaluated by Trypan blue permeability. By comparison to other processing methods, the PCT Shredder in combination with PCT disrupted virtually all nematodes in a heterogeneous culture, whereas when processed by sonication, some larval stage nematodes remained undamaged. Furthermore, temperature fluctuations during processing by thermostated bead beating and sonication resulted in highly variable protein recoveries when compared to the PCT method of processing. In addition, large protein aggregates were observed under a microscope in bead beating and sonication preparations, but were not present in the PCT Shredder preparations. Moreover, protein denaturation and precipitation was observed in bead beating, resulting in the gradual loss of soluble protein over repetitive cycles; this did not occur with the PCT Shredder and PCT.

The tough exterior cuticle of Caenorhabditis elegans makes the nematode very resistant to lysis and impedes proteomic and glycoproteomic analyses. These experiments showed that in relatively mild physiological buffers, nematodes can withstand brief exposure to hydrostatic pressure up to 45,000 psi. Live worms were observed after 20 pressure cycles in which the pressure was sustained at 20,000 psi for 20 seconds during each cycle. After 40 cycles, 100% of the worms were killed, but negligible disruption of the cuticle was observed by microscopy. Damage to adult hermaphrodites evaluated by Trypan Blue staining was minimal, and limited to the expulsion of embryos and some detachment of basement membranes from the cuticle, while dauer stage larvae were more resistant to high pressure. The poor disruption observed by microscopy correlated with minimal protein recovery.

Frequently, downstream analyses requires the preservation of molecular conformation and biological activity of proteins, as well as the stabilization of protein complexes. Chaotropes and detergents alter the native state of proteins and are incompatible with affinity chromatography, ELISA, and immunoprecipitation methods and prohibitive of direct analysis by mass spectrometry. However, without such stringent reagents, total protein yields may be curtailed by an order of magnitude and may also be biased towards hydrophilic proteins. Even when thermostated, temperature fluctuations during bead beating and sonication affects reproducibility and can result in losses from protein aggregation and precipitation. Higher protein yields in physiological buffers were obtained using the PCT Shredder of the present invention.

Materials and Methods

Nematode Cultures

Heterogeneous C. elegans N2 wild type populations (larval through adult hermaphroditic stages) were collected by washing the surfaces of 2% agarose cultures twice with 50 mM K₃PO₄ pH 7.2. The washes were combined and the biomass was pelleted centrifugally. The pellet was additionally washed to remove residual Escherichia coli and the live nematodes were concentrated in an Ultrafree-CL centrifugal filtration device.

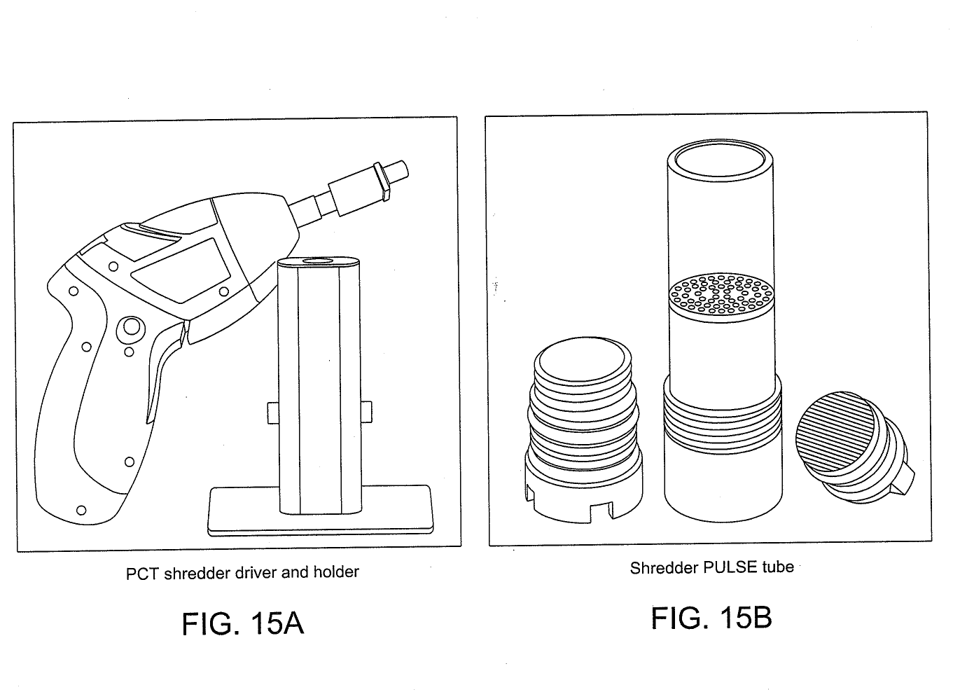

FIGS. 15A and 15B shows the PCT shredder and holder and the shredder PULSE tube.

Pressure Enhanced Processing (PrEP) Kits

The PCT Shredder Kit includes Shredder PULSE Tubes, cordless Shredder Driver, and spring-tensioned Shredder Holder with pressure indicator. The CE PrEP Kit includes the ProteoSOLVE CE Lysis Reagent, ion-exchange resin, ProteoSOLVE Reducing Reagent, and low protein binding abrasive particles. Both kits are available from Pressure BioSciences, South Easton, Mass.

Frozen Abrasive Shredder Technique (FAST)

Fifty milligrams of live worm paste, 100 mg of abrasive particles, and 50 uL of protease inhibitor cocktail from Sigma-Aldrich, St. Louis, Mo., were added to the ram side of the PULSE Tube. As much as 250 mg of paste and 250 mg abrasive can be processed in a single PULSE Tube. A serrated ram was inserted and the completed assembly was vortexed, then lash frozen on dry ice for 5-10 minutes. The PULSE Tube was then engaged in the Shredder Holder and the frozen sample was rotationally ground with the Shredder Driver until the entire sample was expressed through the perforations of the stationary disc.

Pressure Cycling Technology (PCT)

1300 uL of 50 mM K₃PO₄ buffer was added to each PULSE Tube and vortexed. 100 uL of the suspension was reserved as negative control. PCT was performed in the Barocycler NEP 3229, typically for 20-60 cycles at 35,000 or 45,000 psi maximum pressure. Following PCT, the PULSE Tubes were evacuated. Abrasive particles and any nematode debris were pelleted centrifugally at 10,000 RCF for 10 minutes. The supermatants were reserved for protein assay and the pellets were examined microscopically following Trypan Blue staining.

Discussion

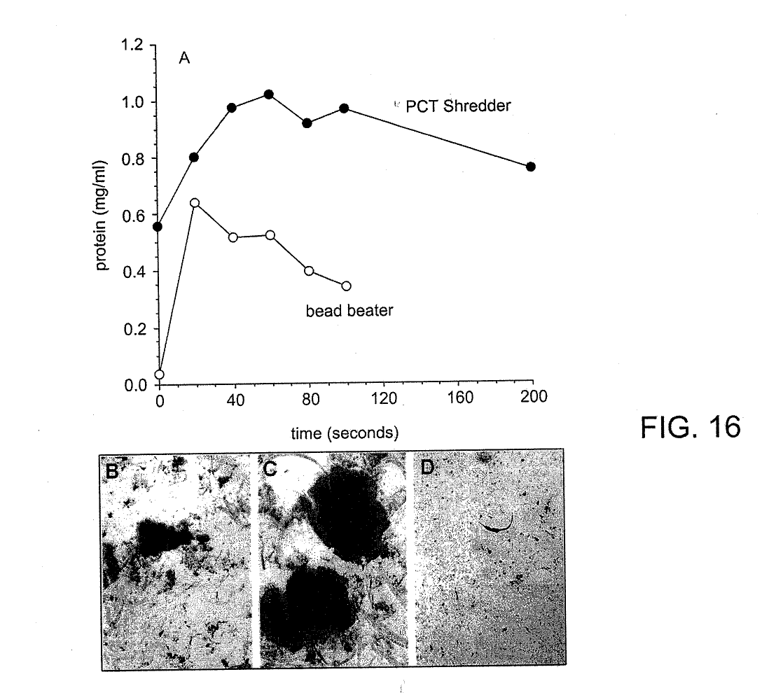

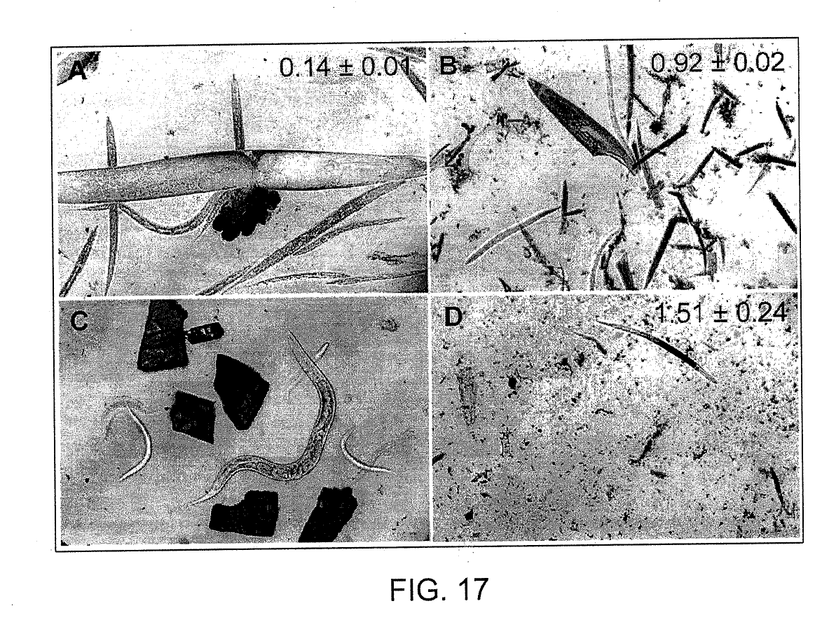

In physiological buffers, nematodes can withstand high pressure as evidenced by Trypan Blue permeability of only 10-14% of nematode cuticles following 20 cycles at 35,000 psi maximum pressure. Adult hermaphrodites were selectively destroyed at this pressure while larvae were more resilient (FIG. 17A). The PCT Shredder disrupted all nematodes including dauer stage larvae (FIG. 17B). In physiological buffers, protein yields were six times greater with the PCT Shredder than with high pressure alone, and an order magnitude higher when the PCT Shredder was used in combination with freezing and abrasives (FIG. 17C and FIG. 17D). Protein yields were more than doubled when the chaotropic ProteoSOLVE CE Lysis Reagent and ProteoSOLVE Reducing Reagent provided in the CE PrEP kit were used (not shown).

By comparison, temperature fluctuations during thermostated bead beating resulted in highly variable protein recoveries. Further, large protein aggregates observed microscopically in bead beating preparations were not observed in the PCT Shredder preparations (FIGS. 16A to 16D). Moreover, protein denaturation was observed in bead beating preparations, resulting in the gradual loss of soluble protein with each successive cycle.

### Example 9

### Improved DNA Recovery from Spinach Leaves Using the PCT Shredder™ and Pressure Cycling Technology (PCT)

The high content of fibrous material in many plant samples as well as the presence of rigid cell walls complicates extraction of DNA from plant tissues. To release target analytes, plant samples often require extensive and time consuming sample disruption by grinding with a mortar and pestle or by homogenizing with glass or metal beads. Such methods are often inefficient and may even be deleterious to the DNA. Here we describe a system for the efficient extraction of DNA from spinach leaves using the PCT Shredder and the Pressure Cycling Technology Sample Preparation System (PCT SPS). Initial disruption of plant tissue with the PCT Shredder followed by DNA extraction by pressure cycling technology (PCT) are carried out in the same processing container (Shredder PULSE Tube). This method of extraction is safe, convenient and efficient. Furthermore, the extracted DNA was far less sheared as compared to DNA extracted by bead beating.

Pressure Cycling Technology (PCT)

In the Pressure Cycling Technology Sample Preparation System (PCT SPS) hydrostatic pressure was rapidly cycled between ambient and ultra high levels (45,000 psi) to control biomolecular interactions. High hydrostatic pressure acts preferentially on the compressible components of the sample, such cell membranes, resulting in cell lysis and the release of intracellular contents. The PCT SPS can be used to disrupt plant and animal tissues, cells, cellular structures and microbes to extract nucleic acids, proteins and lipids. The system is comprised of a small, semi-automated bench-top instrument (Barocycler NEP3229 or the NEP2320) used in combination with single-use sample processing containers (PULSE Tubes). PCT in the presence of suitable extraction reagents, such as DNAzol®, results in isolation of intact DNA for genomic analysis and other applications.

The PCT Shredder

The PCT Shredder was designed to physically disrupt and enhance extraction of tough, fibrous and other difficult-to-disrupt biological materials such as certain plant and animal tissues. The PCT Shredder was used to rapidly grind the sample directly in a specially designed Shredder PULSE Tube to increase the tissue surface area and to improve cell lysis prior to treatment by PCT for extraction of nucleic acids, proteins, lipids and other cellular contents. Since shredding and PCT are done in the same tube, loss of sample or the likelihood of cross contamination is significantly reduced as compared to other processing methods.

Materials and Methods

A comparison was made of samples processed by the PCT Shredder followed by PCT, PCT alone, or bead beating. For each condition, approximately 200 mg of fresh baby spinach leaves were chopped or torn into pieces (excluding midveins). One set of samples was processed with the PCT Shredder in the presence of 0.7 mL of DNAzol® (Invitrogen) for 20 seconds at ambient temperature. After shredding, the sample volume was brought up to 1.4 mL with additional DNAzol® reagent. The Shredder Pulse Tube was then capped with a high pressure Shredder PULSE Tube cap provided with the PCT Shredder Kit and subjected to PCT (35,000 or 45,000 psi held for 20 seconds, followed by atmospheric pressure held for 10 seconds and repeated for 30 cycles) at ambient temperature. A second set of samples was processed by PCT alone without pre-processing with the PCT Shredder. These samples were loaded into standard FT500 PULSE Tubes with 1.4 mL of DNAzol® and subjected to PCT as described above. A third Set of samples was subjected to bead beating in a Mini-beadbeater-1 (BioSpec Products) using 1 mL of 1.0 mm Zirconia beads in a 2 mL centrifuge tube. Samples were disrupted by bead beating at full power using ten 10 second bursts. Because of the heat generated during bead beating, samples were cooled on ice between bursts. After extraction by PCT or bead beating, DNA was purified using the DNAzol® isolation protocol for plants according to manufacturer's instructions. DNA recovery was measured by Qubit assay using the Quant-iT dsDNA BR kit (Invitrogen). DNA was visualized by agarose gel electrophoresis using the Reliant FastLane Ge System (Lonza).

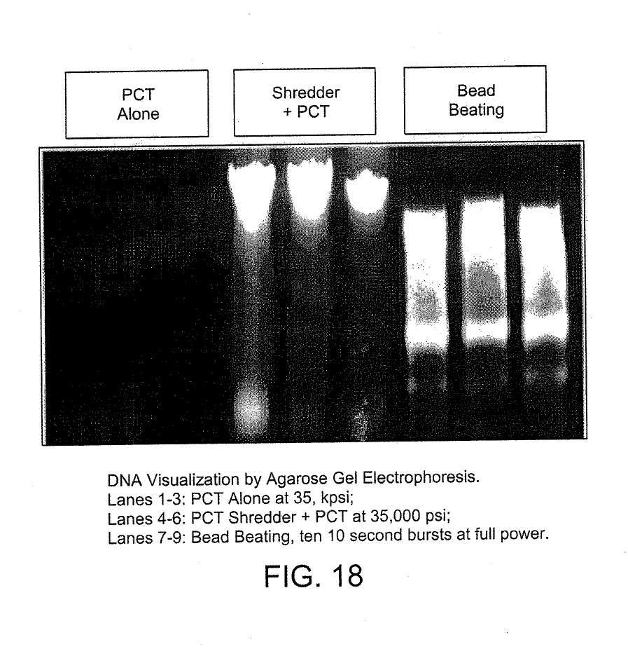

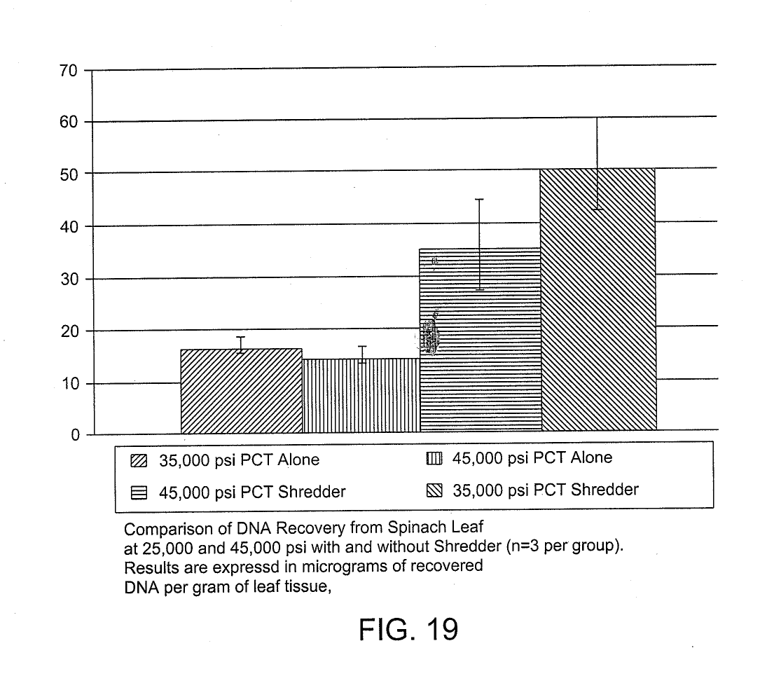

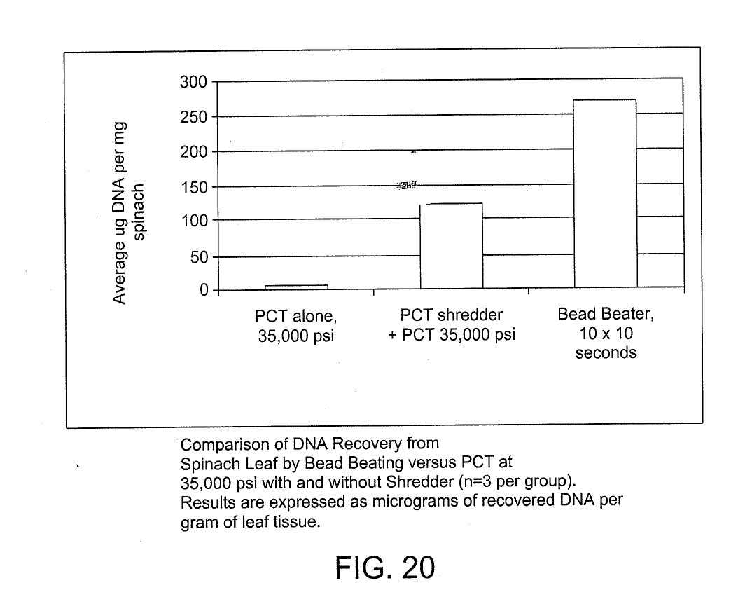

Three methods of DNA extraction of DNA from spinach leaves were compared in this study. Specifically, yield and quality of DNA were evaluated for samples processed by the PCT Shredder in combination with PCT, PCT alone, and bead beating. Data show that DNA recovery from samples processed by the combination of the PCT Shredder followed by PCT was significantly higher than from samples processed by PCT alone (See FIGS. 19 and 20). Interestingly, in these experiments, more DNA was recovered from samples processed at 35,000 psi than from samples processed at 45,000 psi (FIG. 19), indicating that higher pressure is not necessary to release high quality DNA from this sample. However, it may be possible that 45,000 psi may actually be detrimental to extraction of DNA from spinach. Therefore, in all subsequent experiments, PCT was performed at 35,000 psi. Extraction by the PCT Shredder followed by PCT was also compared to bead beating, a common method for the extraction of DNA from plants. Although samples disrupted by bead beating averaged ˜270 μg of DNA per gram of spinach as compared to ˜120 μg of DNA per gram of spinach from the combination of the PCT shredder and PCT treatment at 35,000 psi, the DNA recovered from the bead beating process was significantly more sheared and fragmented than DNA obtained from the combination of the PCT Shredder and PCT (See FIG. 18).

Conclusions

The combination of the PCT Shredder followed by pressure cycling technology (PCT) can be an effective and safe method for extraction of DNA from samples such as spinach. Efficient recovery of high quality DNA can be obtained for sequencing, cloning or other experiments. Since disruption and extraction are performed in the same container (Shredder Pulse Tube), the possibility of sample loss due to multiple transfers is reduced. In addition, the likelihood of cross-contamination is minimized. Although more total DNA was obtained using bead beating, the quality of the DNA, as assessed by average length and degree of fragmentation, was significantly lower than the DNA obtained by using the combination of the PCT Shredder and the Pressure Cycling Technology Sample Preparation System.

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. For example, rotation can be performed in a single direction or reversibly in an aperiodic or periodic frequency.

Those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

Moreover, it should also be appreciated that the invention is directed to each feature, system, subsystem, or technique described herein and any combination of two or more features, systems, subsystems, or techniques described herein and any combination of two or more features, systems, subsystems, and/or methods, if such features, systems, subsystems, and techniques are not mutually inconsistent, is considered to be within the scope of the invention as embodied in the claims. Further, acts, elements, and features discussed only in connection with one embodiment are not intended to be excluded from a similar role in other embodiments. For example, rotation can be performed at a variety or plurality of rotational rates, directions, and at various combinations of pressure conditions.

As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims.

What is claimed:

1. A device for sample processing, comprising a container and a rotating element configured to interface with a rotational drive

2. The device of claim 1, wherein the rotating element is configured to interface with a rotational driver selected from the group consisting of an electrical motor, a hand crank, and, a pressure-generating device configured to exert pressure on the sample.

3. The device of claim 1, further comprising a smooth perforated divider or a perforated divider with sharp surface features, such as serration, perforation or teeth which facilitate homogenization.

4. The device of claim 1, wherein the rotating element has sharp surface features, such as serration, perforation or teeth, which provide the grip necessary to rotate a solid sample block against a grinding surface.

5. The device of claim 1, wherein the rotating element serves as a plunger (ram) transmitting hydrostatic pressure into the sample container.

6. The device of claim 1, without lysis disk and comprising grinding aids such as small balls are added to the tube.

7. The device of claim 1 further comprising a plurality of at least one of grinding balls and beads disposed in the container.

8. The device of claim 1 further comprising a cap configured to allow venting of any air.

9. The device of claim 1 wherein the amount of force exertable upon the rotating element is controlled by a spring.

10. The device of claim 1 further comprising an electromechanical device comprising motor and a regulator that is configured to control the speed of rotation of the rotatable element.

11. The device of claim 1 wherein the frequency and the amplitude of shaking agitation is controlled by a reciprocal shaker device, if small balls of beads are utilized.

12. The device of claim 1 further comprising a liquid selected from the group consisting of a lysis buffer and extraction solvent is disposed with a sample in the container.

13. The device of claim 1 further comprising a plurality of perforated discs having surface features of varying size, the discs disposed in the container.

14. The device of claim 1 wherein a series of serrated plungers with surface features of varying size is used consecutively in the same sample container.

15. The device of claim 1 wherein the sample containers is a single use tube.

16. The device of claim 1 wherein the containers are reusable.

17. The device of claim 1 wherein the sample containers are comprised of a polymeric material or a metal.

18. The device of claim 1 wherein the sample containers are made of stainless steel.

19. The device of claim 1 wherein balls or beads of multiple sizes are disposed concurrently in the container.

20. A method of preparing a sample, comprising:
charging a sample into a sample container, and
rotating a rotatable element having a surface thereof disposed against the sample.

21. The method of claim 20 further comprising applying a hydrostatic pressure on the sample within the container.

22. The method of claim 21, wherein the applied hydrostatic pressure is generated by reducing the volume contained within the container by axially displacing the rotatable element and compressing the container internal volume.

23. The method of claim 22, further comprising cooling the sample.

24. The method of claim 22, further comprising heating the sample while contained in the sample container.

25. The method of claim 22, further comprising utilizing a sample container having a lysis disk disposed therein.

26. The method of claim 20, further comprising charging at least one of abrasive media and grinding aids into the sample container.

27. The method of claim 20, further comprising agitating the sample within the sample container with a shaker device.

28. The method of claim 20, further comprising rotating the rotatable element at a predetermined rate of revolutions.

29. The method of claim 20, further comprising cyclically rotating the rotatable element.

30. The method of claim 20, further comprising rotating the rotatable element at a first rotating rate and rotating the rotatable element at a second rotating rate.
